(19) United States
(12) Patent Application Publication
(10) Pub. No.: US 2004/0161419 A1
(43) Pub. Date: Aug. 19, 2004

(54) Placental stem cells and uses thereof

(76) Inventors: Stephen C. Strom, Allison Park, PA (US); Toshio Miki, Pittsburgh, PA (US)

Correspondence Address: Deborah A. Somerville, KENYON & KENYON, One Broadway, New York, NY, 10004, US

(21) Appl. No.: 10/691,468

(22) Filed: Oct. 22, 2003

Related U.S. Application Data
(63) Continuation-in-part of application No. 10/420,656, filed on Apr. 21, 2003.
(60) Provisional application No. 60/374,172, filed on Apr. 19, 2002.

(51) Int. Cl. A61K 48/00; C12N 5/08

(52) U.S. Cl. 424/93.21; 435/366

(57) ABSTRACT

The present invention features novel placental stem cells and provides methods and compositions for the therapeutic uses of placental stem cells or placental stem cells that have been induced to differentiate into a desired tissue type into a recipient host in amounts sufficient to result in production of the desired cell type, e.g, hepatocytes, neural cells, pancreatic cells, vascular endothelial cells, cardiomyocytes.

### CROSS REFERENCE TO RELATED APPLICATIONS

This application claims the benefit of U.S. application Ser. No. 10/420,656 filed Apr. 21, 2003, which claims priority to U.S. Provisional Application Serial No. 60/374,172 filed Apr. 19, 2002.

### BACKGROUND OF THE INVENTION

Embryonic stem cells have long been recognized as a source of totipotent stem cells, able to give rise to different cell types. These cells are derived from the inner cell mass of fertilized and developing embryos. The use of such cells has been controversial on both ethical and religious grounds. Furthermore, federal regulation currently limits the use of embryonic stem cells to a few established cell lines which are difficult to obtain. Recent studies have focused on alternative sources of stem cells. These include hematopoietic stem cells obtained from bone marrow or peripheral blood. However the isolation of such stem cells from individuals can be invasive and painful.

The developing embryo requires that the interaction with the mother be mediated by the placenta and extraembryonic membranes. The placenta and chorion is derived from the trophoblast, which begins to differentiate from the inner cell mass as early as day 8 following fertilization while the amniotic cavity originates in the ectoderm of the inner cell mass and consists of a single layer of extraembryonic mesoderm.

In recent years, the placenta, the amnion and cord blood have been studied as alternative sources of stem cells. Fetal mesenchymal cells and mesenchymal amniocytes have been isolated from both the human placenta and amniotic fluid for use in fetal tissue engineering in surgical reconstruction of severe birth defects. Immunocytochemistry of these cells demonstrated the expression of markers such as calponin, desmin, SMA, cytokeratin-8 and cytokeratin-18. These cells were further probed for their ability to attach and proliferate on implantable, biodegradable scaffolds (Kaviani et al., J. Pediatr Surg. 36:1662-1665 (2001) and Kaviani et al., J. Pediatr Surg. 37:995-999 (2002)). Fetal mesenchymal stem cells have also been isolated from second trimester amniotic fluid (In't Anker et al., Blood. 102(4):1548-9 (2003). Additionally, amniotic fluid samples collected from amniocentesis procedures were also found to contain cells that express the pluripotent stem cell marker, Oct-4 (Prusa et al. Human Reproduction. 18(7):1489-1493 (2003)).

Embryonic-like stem cells have also been collected by perfusing the placenta with solutions containing anticoagulants to flush out residual cells from areas of the placenta that are vascularized. In the following patent applications US 2002/0123141 entitled “Method of Collecting Placental Stem Cells”, US 2003/0032179 entitled “Post-Partum Mammalian Placenta, Its Use and Placental Stem Cells Therefrom”, US 2003/0180269 and WO 03/068937 both entitled “Embryonic-like Stem Cells Derived From Post-Partum Mammalian Placenta and Uses and Methods of Treatment Using Said Cells”, Hariri reports that the first collection of blood from the perfused placenta, referred to as cord blood, contains populations of hematopoeitic progenitor cells which are CD34 positive and CD38 positive or CD34 positive and CD38 negative or CD34 negative and CD38 positive. Subsequent perfusions of the placenta were reported to yield embryonic-like stem cells that are SSEA-3 negative, SSEA-4 negative, Oct-4 positive, ABC-p positive, CD10 positive, CD38 negative, CD29 positive, CD34 negative, CD44 positive, CD45 negative, CD54 positive, CD90 positive, SH2 positive, SH3 positive and SH4 positive.

Amniotic epithelial (AE) cells have been isolated from the amnion and were initially examined for their ability to synthesize large quantities of lysosomal enzymes in vitro that are lacking in patients with certain enzymatic disorders (Sakuragawa et al., Cell Transplantation 4:343-346 (1995)). AE cells that were isolated by Akle et al (The Lancet 1003-1005 (1981)) were found to not express HLA-1, B, C and DR antigens or beta 2-microglobulin. The absence of several classes of MHC on the surface of AE cells suggested that these cells may be implanted in patients and indeed grafts of amniotic tissue were tolerated by volunteers for up to 54 days without evidence of rejection. However clinical trials of amniotic tissue transplantation that were subsequently carried out in patients with inborn errors of metabolism did not produce a definitive clinical benefit (Scaggiante et al., Transplantation 44: 59-61 (1987)).

A significant problem with the use of these AE cells in transplantation was the limited number of AE cells that were obtainable from a donor. To induce proliferation of AE cells, Tohyama et al transfected these cells with SV40 Large T antigen. Although the cell line proliferated, it was reported to only have limited therapeutic value. In fact, the cells were found to be tumorigenic upon transplantation (Tohyama et al., Tohoku J. Med. 182:75-82 (1997)). Other approaches to culturing AE cells included supplementing a basal media with hepatocyte growth factor (HGF, 50 ng/ml) or epidermal growth factor (EGF, 50 ng/ml). The addition of these growth factors reportedly increased the number of cells in an initial culture 2 to 7 fold. However after 11 days of culture, the cells were reported to cease proliferation (Terada et al., Cell Transplantation 9:701-704 (2000)).

Hu et al. (WO00/73421 entitled “Methods of Isolation, Cryopreservation and Therapeutic Use of Human Amniotic Epithelial Cells”) reported the isolation, culturing and cryopreservation of amniotic epithelial cells (termed “multipotential cells”). These cells were characterized by round cobblestone morphology, large nuclei, epithelial membrane antigen and cytokeratin staining, and gap junctional communication. These investigators disclose culturing AE cells in various media such as DMEM, F12, M199 and RPMI that could be supplemented with fetal bovine serum, whole human serum or human umbilical cord serum collected at the time of delivery, or supplemented with growth factors, cytokines, hormones, or any combinations thereof. Hu et al. further report that the multipotentiality of the AE cells may be demonstrated by their ability to form teratomas after injection into nude or SCID mice. They however, did not characterize their AE cells for the expression of any embryonic stem cell, or differentiated stem cell markers.

Besides enzyme replacement therapy, AE cells and membranes have also been investigated for use in restoring epithelialization of corneal surfaces in patients, dressings or skin grafts in the treatment of dermal abrasions, and severe burns. For example, Sackier et al, isolated amniotic epithelial cells and applied them using clinical procedures for the treatment of diseased or damaged tissues in joints denuded of cartilage and vascular grafts (U.S. Pat. No. 5,612,028 entitled “Method of regenerating or replacing cartilage tissue using amniotic cells”, EP333328 entitled “Clinical developments using amniotic cells”). Kobayashi et al. (Cornea 21:62-67 (2002)) isolated and cultured predominantly cytokeratin-positive amniotic epithelial and mesenchymal cells from human amniotic membranes. The cell culture supernatant was reported to contain potent inhibitors of neovascularization.

In further experiments, Sakuragawa et al. (Neuroscience Lett. 209:9-12 (1996)) showed that amniotic epithelial (AE) cells express neuronal markers such as RC1, A2B5, CNPase, vimentin, neurofilament protein, microtubule associated protein 2, microtubule associated protein 2 kinase, glial fibrilliary acidic protein, myelin basic protein, galactocerebroside and cyclic nucleotide phosphodiesterase. These cells were cultured in RPMI-1640 medium supplemented with 10% fetal calf serum. Later studies also indicated that the AE cells express choline acetyltransferase mRNA and synthesize and release acetylcholine (Sakuragawa et al. Neurosci Lett. 232:53-56 (1997), EP815867 entitled “Cells for Treating Dementia”) and catecholamines (Elwan and Sakuragawa. Neuroreport 8: 3435-3438 (1997)). These cells were further described as being useful in the treatment of dementia (EP815867).

Recently, Sakuragawa (US 2003/0044977 entitled “Human Stem Cells Originated From Human Amniotic Mesenchymal Cell Layer”) reported the isolation of human stem cells from the human amniotic mesenchymal cell layer. These amniotic mesenchymal cells express vimentin and nestin which are markers of neural stem cells. In addition, Sakuragawa noted that neural stem cells are not present in the amniotic epithelial cells.

Evidence of AE liver cell specific protein expression has also been reported. For example, cultured AE cells were shown to be immunoreactive with antibodies to human albumin and alpha-fetoprotein in vitro and also following transplantation into mouse liver (Sakuragawa et al., J Hum Genet. 45:171-176 (2000)). However, AE cells that are cultured in the conditions described in Sakuragawa et al. J Hum Genet. 45:171-176 (2000) and Sakuragawa et al. Neuroscience Lett. 209:9-12 (1996) (e.g. in the absence of EGF 10 ng/ml) do not proliferate well. Clear differences between the cultured AE cells of Sakuragawa (J Hum Genet. 45:171-176 (2000) and EP815867) and the cultured placental stem cells of this invention may be observed in FIGS. 2, 3 and 7. Example 3, Tables 3-5 also further distinguish the placental stem cells of this invention from previously described AE cells isolated by Sakuragawa.

### SUMMARY OF THE INVENTION

The present invention features novel placental stem cells. Preferred cells are obtained from a human placenta. Particularly preferred placental stem cells express at least one and preferably at least two biomarkers selected from the group consisting of: c-kit, Thy-1, OCT-4, SOX2, SSEA3, SSEA4, TRA1-60, TRA1-81, Lefty A, FGF-4, Rex-1, and TDGF-1. Other preferred cells are negative for expression of CD34. Particularly preferred cells were deposited with the American Type Culture Collection on ______ and have been assigned ATCC accession number ______.

In another aspect, the invention provides methods for culturing placental stem cells for propagation and/or differentiation into specific cell types including but not limited to pancreatic cells, neural cells, vascular endothelial cells, cardiomyocytes and hepatocytes.

In one embodiment, the cells are cultured under appropriate conditions and for a sufficient period of time to differentiate into hepatocytes. Examples of appropriate conditions include culturing in media, which is supplemented with type I collagen, EGF (10 ng/ml), dexamethasone (0.1 μM), insulin (10 μg/ml), transferrin (5.5 μg/ml), selenium (6.7 ng/ml), ethanolamine (2 μg/ml) and phenobarbital (1 mM). Preferred hepatocytes express at least one marker and preferably at least two markers selected from the group consisting of: albumin, CYP3A4, A1AT, HNF1, HNF4 and C/EBP-alpha. Particularly preferred hepatocytes were deposited with American Type Culture Collection on ______ and have been assigned ATCC accession number ______. An effective amount of hepatocytes so derived may be administered to a subject to treat a liver disease or disorder. Alternatively, the hepatocytes or placental stem cells may be used to generate a bioartificial liver, which can be implanted into a subject to provide liver cell factors that are needed to treat the subject for a liver disease or disorder. For example, the hepatocytes may be introduced into an animal liver to “humanize” the animal liver. In addition to therapeutic uses, the hepatocytes or bioartificial livers may be useful for screening drugs for liver toxicity. For example, hepatocytes or bioartificial livers may be incubated with defined concentrations of drugs for defined times and the biological effects measured.

In another aspect, placental stem cells are cultured under appropriate conditions and for a sufficient period of time to differentiate into vascular endothelial cells. Examples of appropriate conditions include culturing in Matrigel™. Particularly preferred vascular endothelial cells were deposited with American Type Culture Collection on ______ and have been assigned ATCC accession number ______. An effective amount of the vascular endothelial cells may be administered to a subject with a vascular disease or disorder to treat the vascular disease or disorder.

In another aspect, placental stem cells are cultured under appropriate conditions and for a sufficient period of time to differentiate into pancreatic cells. Examples of appropriate conditions include culturing in media, which is supplemented with pancreatic cell differentiation factors such as dexamethasone (0.1 μM), insulin-transferrin-selenium (ITS) or culturing in Matrigel™. Preferred pancreatic cells express at least one marker and preferably at least two markers selected from the group consisting of: Nkx-2.2, glucagon, Pax6, Pdx1 and insulin. Particularly preferred pancreatic cells were deposited with American Type Culture Collection on ______ and have been assigned ATCC accession number ______. An effective amount of pancreatic cells may be administered to a subject with a pancreatic disease or disorder to treat the pancreatic disease or disorder.

In a further aspect, placental stem cells are cultured under appropriate conditions and for a sufficient period of time to differentiate into neural cells. Examples of appropriate conditions include culturing in a media, which is supplemented with all-trans retinoic acid or FGF-4 (10 ng/ml). Preferred neural cells express at least one marker and preferably at least two markers selected from the group consisting of: C-type natriuretic peptide (CNP) neuron specific enolase (NSE), neurofilament-M (NF-M), myelin basic protein (MBP), glial fibrillary acid protein (GFAP), nestin and glutamic acid decarboxylase (GAD). Particularly preferred neural cells were deposited with American Type Culture Collection on ______ and have been assigned ATCC accession number ______. An effective amount of the neural cells may be administered to a subject with a neural disease or disorder to treat the neural disease or disorder.

In yet a further aspect, placental stem cells are cultured under appropriate conditions and for a sufficient period of time to differentiate into cardiomyocytes. An example of appropriate conditions include culturing in media, which is supplemented with L-ascorbic acid 2-phosphate (1 mM). Preferred cardiomyocytes express at least one marker and preferably at least two markers selected from the group consisting of: cardiac transcription factor 4 (GATA-4), cardiogenic homeodomain factor (Nkx 2.5), atrial myosin light chain type 2 (MLC-2A), ventricular myosin light chain type 2 (MLC-2V), human atrial natriuretic peptide (hANP), cardiac troponin T (cTnT), cardiac troponin I (cTnI), or alpha-actinin. Particularly preferred cardiomyocytes were deposited with American Type Culture Collection on ______ and have been assigned ATCC accession number ______. An effective amount of the cardiomyocytes may be administered to a subject with a cardiac disease or disorder to treat the cardiac disease or disorder.

Placental stem cells provide a noncontroversial source of stem cells that can be differentiated into a variety of cells and tissue types, including liver, pancreas, endothelial, neural and cardiac muscle cells and tissues. Other features and advantages of the invention will be apparent from the following Detailed Description and claims.

### BRIEF DESCRIPTION OF THE DRAWINGS

FIG. 1 is a bar graph showing that the cultured placental stem cells express characteristic embryonic stem cell surface markers: stage specific embryonic antigen 3 and 4 (SSEA-3, SSEA-4); tumor related antigen 1-60 (TRA 1-60); TRA 1-81, thymidylate synthase complementing protein (Thy-1) and the proto-oncogene tyrosine-protein kinase kit (c-kit).

FIG. 2 is a growth curve showing that placental stem cells grow significantly better in the presence of Epidermal Growth Factor (EGF) (10 ng/ml (square)) than in the absence of EGF (10 ng/ml (circle)).

FIG. 3 is a light micrograph showing placental stem cells cultured for 14 days in the absence (w/o EGF) or presence of EGF 10 ng/ml.

FIG. 4 is a gel showing the placental stem cells cultured for 0 and 24 days with Epidermal Growth Factor (EGF) 10 ng/ml continue to express characteristic stem cell markers: Octamer-binding transcription factor-4 (also known as OCT-3/4); Sex determining region Y related-HMG box 2 (SOX2); left-right determination factor A (Lefty-A); fibroblast growth factor 4 (FGF-4); Rex-1 (also known as zinc finger protein-42 (ZFP-42)) and teratocarcinoma-derived growth factor-1 (TDGF-1).

FIG. 5 are micrographs showing phase contrast images (A, C, E, G, I) and immunofluorescent images (B, D, F, H, J) of embryoid body (EB) like structures formed by culturing placental stem cells to 80% confluence in media containing 10% Fetal Bovine Serum and EGF 10 ng/ml prior to transferring such cells onto a 20% (v/v) Matrigel coated plate; FIG. 5(B) shows immunohistofluorescent staining of placental stem cells with antibodies against alkaline phosphatase; FIG. 5(D) shows immunohistofluorescent staining of placental stem cells with antibodies against stage specific embryonic antigen antibody-3 (SSEA-3); FIG. 5(F) shows immunohistofluorescent staining of placental stem cells with antibodies against stage specific embryonic antigen antibody-4 (SSEA-4); FIG. 5(H) shows immunohistofluorescent staining of placental stem cells with antibodies against tumor related antigen 1-60 (TRA 1-60); FIG. 5(J) shows immunohistofluorescent staining of placental stem cells with antibodies against tumor related antigen 1-81 (TRA 1-81).

FIG. 6 are micrographs showing immunohistochemical staining of human placental tissue (left panel) and placental stem cells (right panel) with antibodies against cytokeratin AE1/AE3, cytokeratin 19 (CK19), cytokeratin 18 (CK18), the proto-oncogene tyrosine-protein kinase kit (c-kit), thymidylate synthase complementing protein (Thy-1), alpha-1-antitrypsin (A1AT), and alpha fetoprotein (AFP).

FIG. 7 is a bar graph showing the relative differences in RNA expression of various liver-specific markers in the placental stem cells of the present invention as compared to those described in Sakuragawa et al (Sakuragawa et al., J Hum Genet. 45:171-176 (2000)).

FIG. 8(A) is a bar graph showing the induction of hepatocyte specific mRNA (albumin, alpha-1-antitrypsin (A1AT), and C/EBP-alpha) in placental stem cells cultured for 0, 3, 9 and 15 days on Type-I collagen coated plates supplemented with dexamethasone (0.1 μM), insulin (0.1 μM) and phenobarbital (1 mM); FIG. 8(B) are micrographs showing immunohistochemistry staining of hepatocytes derived from placental stem cells using antibodies against human albumin (upper panels), and antibodies against hepatocyte nuclear factor-4 alpha (HNF-4 alpha) (lower left panel). The lower right panel shows a phase contrast image of hepatocytes derived from placental stem cells; FIG. 8(C) is a bar graph showing that hepatocytes derived from placental stem cells exhibit cytochrome P450 (CPY1A1/CPY1A2) activity upon beta-naphthoflavone (50 μM) induction at levels that are approximately 60% of the activity of freshly isolated human hepatocytes. CPY1A1/CPY1A2 activity was measured using an ethoxyresorufin-o-deethylase (EROD) assay; FIG. 8(D) is a chromatogram of a high pressure liquid chromatographic (HPLC) separation of testosterone metabolite, 6-beta-hydroxy testosterone, generated in hepatocytes derived from placental stem cells.

FIG. 9(A) are fluorescent micrographs showing placental stem cells expressing glial fibrillary acid protein (GFAP), C-type natriuretic peptide (CNP) and beta-tubulin III; FIG. 9(B) is a gel showing the placental stem cells cultured for 0 and 7 days in media supplemented with all-trans retinoic acid express neural specific markers such as nestin, neuron specific enolase (NSE), neurofilament-M (NF-M), glutamic acid decarboxylase (GAD), glial fibrillary acid protein (GFAP), and myelin basic protein (MBP).

FIG. 10 are light and electron micrographs showing vascular endothelial cells generated from placental stem cells cultured on Matrigel™.

FIG. 11 is a gel showing that placental stem cells cultured for 14 days in media supplemented with nicotinamide (10 mM) express pancreatic cell specific markers such as insulin, glucagon, homeobox transcription factor Nkx-2.2, paired box gene 6 (Pax6) and pancreatic duodenal homeobox 1 (Pdx1).

FIG. 12(A) is a gel showing that placental stem cells cultured for 0 and 14 days in media supplemented with ascorbic acid 2 phosphate (1 mM) express cardiac specific markers such as cardiac transcription factor-4 (GATA-4), cardiogenic homeodomain factor Nkx 2.5, atrial myosin light chain type 2 (MLC-2A), ventricular myosin light chain type 2 (MLC-2V), human atrial natriuretic peptide (hANP), and cardiac troponin T (cTnT); FIG. 12(B) is an immunofluorescent micrograph showing actinin expression in cardiomyocytes derived from placental stem cells.

### DETAILED DESCRIPTION OF THE INVENTION

1. General

The present invention features novel placental stem cells that have been obtained from the amnion, chorion and decidual layers of the placenta. Exemplary cells were deposited with American Type Culture Collection, 10801 University Blvd. Manassas, Va. 20110-2209 on ______ and have been assigned ATCC accession number ______. The placental stem cells of the invention express at least one and preferably at least two markers normally associated with embryonic stem cells including but not limited to proto-oncogene tyrosine-protein kinase kit (c-kit, also known as CD 117 or mast/stem cell growth factor receptor precursor), thymidylate synthase complementing protein (Thy-1); Octamer-binding protein ¾ (OCT-¾); Sex determining region Y-box 2 (SOX2); stage-specific embryonic antigen 3 (SSEA3); stage-specific embryonic antigen 4 (SSEA4), tumor related antigen 1-60 (TRA1-60); TRA1-81; left-right determination factor A (Lefty-A); fibroblast growth factor 4 (FGF-4); Rex-1 (also known as zinc finger protein-42 (ZFP-42)) and teratocarcinoma-derived growth factor-1 (TDGF-1). The placental stem cells of the invention can form embryoid bodies (EB) like spheroid structures similar to those formed by embryonic stem cells (See FIG. 5).

In addition, under appropriate conditions, these placental stem cells can differentiate into a variety of cell types including but not limited to hepatocytes, pancreatic cells, neural cells, cardiomyocytes and vascular endothelial cells. Appropriately, differentiated cells are particularly useful to restore function in diseased tissues, for example via transplantation therapy or tissue engineering, and to study metabolism and toxicity of compounds in drug discovery efforts.

2. Placental Stem Cells

a) Growth and Culture

Placental stem cells can be isolated from the amniotic membrane and associated mesenchyme using techniques known to those skilled in the art. For example, amniotic cells may be aspirated from amniotic fluid. Alternatively, the amniotic tissue may be dissected free of chorion and other placental tissues. The amnion layer may be gently stripped from the underlying chorion layer using forceps and a sterile scalpel, disaggregated mechanically and/or treated with digestive enzymes and/or chelating agents that weaken the connections between neighboring cells, making it possible to disperse the tissue suspension of individual cells. The chorion or decidua of the placenta can also be used as a source of placental stem cells.

For example, enzymatic dissociation can be carried out by treating the amnion layer with any of a number of digestive enzymes. Such enzymes include, but are not limited to, trypsin, chymotrypsin, collagenase, elastase and/or hylauronidase. Example 2 describes the treatment and isolation of amniotic tissue with trypsin to dissociate individual cells.

Single cell suspensions can be cultured in medium containing a basal medium, supplemented with serum, hormones, growth factors such as fibroblast growth factors (FGFs), epidermal growth factor (EGF), transforming growth factor-β (TGF-β), platelet derived growth factors (PDGF-AA, PDGF-AB, PDGF-BB), vascular endothelial growth factors (VEGF) and hepatocyte growth factor (HGF); cytokines such as oncostatin M, fms-like tyrosine kinase-3 ligand (Flt-3 ligand), stem cell factor (SCF), thrombopoietin (Tpo), interleukins (IL-3, IL-7, IL-11), colony stimulating factors; antibiotics; trace elements and other additives such as insulin, transferrin, selenium (ITS), glucose, interleukin 6 and histone deacetylase inhibitors such as sodium butyrate or tricostatin A. To induce demethylation or dedifferentiation, 5-azacytidine and/or bone morphogenic protein (BMP) inhibitors may also be added to the medium. Example 2 describes a culture medium that may be used to culture placental stem cells. Those of skill in the art will also recognize that one or more commercially available substances may be used as additives or substitutions to the medium to support the growth of stem cells.

The cells may be plated on tissue culture dishes as shown in Example 2 or may be grown in a cell suspension in a flask, forming spheroidal cell bodies. When grown on tissue culture dishes, the surface may be coated electrostatically or with extracellular matrix components. Cells may be passaged before reaching confluency on the dish to avoid contact inhibition and maintain proliferating growth conditions.

Additionally, cells can be grown by culture with placental stromal cells to promote cell expansion or co-culture with progenitor or differentiated cells derived from different organs and tissue to promote proliferation or differentiation.

In addition, the cells may be grown on feeder layers. In culturing the cells of the invention, it is believed that the use of feeder cells, or an extracellular matrix derived from feeder cells, provides one or more substances necessary to promote the growth of the stem cells and/or inhibits the rate of differentiation. Such substances are believed to include membrane-bound and/or soluble cell products that are secreted into the surrounding medium by the cells. For example, placental stem cells can be grown on a substrate selected from the group consisting of mouse embryo fibroblast cells such as STO cells (e.g. ATCC CRL 1503), human fibroblasts, or human epithelium cells.

b) Cryopreservation of Placental Stem Cells

Placental stem cells may be cryopreserved and thawed with no discernable loss of function. Placental stem cells may be isolated as described and cultured in basal media for 7-10 days or until the cultures grow to confluence. Cells may be trypsinized, washed once to remove trypsin and counted. Placental stem cells may then be cryopreserved by suspending the isolated cells in basal media (90%) supplemented with dimethylsulfoxide (DMSO) (10% v/v) and placing them in a cell freezer container which when placed into a −80 degree C. freezer to cool the cells at a rate of approximately one degree C. per minute. Cells may be stored at −80 C until needed.

Before use, cells can be thawed rapidly by placing the vials in a water bath prewarmed to 37 degrees C. Upon complete thawing, cells are decanted from the cryovials and added to at least 3 volumes of pre-warmed (37 degrees C.) basal media. Cells can then be centrifuged at 100×g for 5 minutes and resuspended in basal media. Cells can be counted at this step, checked for viability and plated on regular culture dishes. Cell viability of the thawed cells may range from 70-95% in different frozen batches of placental stem cells. This is a standard cryopreservation technique used by many cell culturists. Glycerol may be used in place of DMSO at a concentration ranging from 5-40%, DMSO may be used at concentrations ranging from 5-35%, and different media may be substituted for the basal media used here. Different media could include but are not limited to balanced salts solution such as Hank's Balanced Salt Solution (HBSS), any complete tissue culture media such as Minimal Essential Medium (MEM), Dulbecco's Minimal Essential Medium (DMEM), Ham's Medium F12, etc. Cryopreservation solutions may consist of any solution used for the cold storage and transportation of organs from transplantation such as Belzer's UW solution or HKT or an equivalent. The cryopreservation rate of approximately 1 degree per minute is a standard rate but the cryopreservation results may be improved by using different rates allowable through the use of a programmable cell freezer. Cells recovered from cryopreservation attach to culture plates and grow at a rate not discernibly different from cells not previously frozen.

c) Purification and Enrichment of Placental Stem Cells

If needed, cell surface markers such as SSEA3, SSEA4, TRA1-60, TRA1-81, Thy-1, and c-kit may be used to purify enriched populations of cells using a variety of methods. Such procedures involve a positive selection, such as passage of sample cells over a column containing anti-SSEA3, anti-SSEA4, anti-TRA1-60, anti-TRA1-81, anti-Thy-1 or anti-c-kit antibodies or binding of cells to magnetic bead conjugated anti-SSEA3, anti-SSEA4, anti-TRA1-60 anti-TRA1-81, anti-Thy-1 or anti-c-kit or by panning on anti-SSEA3, anti-SSEA4, anti-TRA1-60, anti-TRA1-81, anti-Thy-1 or anti-c-kit antibody coated plates and collecting the bound cells. Alternatively, the single cell suspension may be exposed to a labeled antibody that immuno-specifically binds to the SSEA3, SSEA4, TRA1-60, TRA1-81, Thy-1 or c-kit cell surface antigen. Following incubation, with the SSEA3, SSEA4, TRA1-60, TRA1-81, Thy-1 or c-kit antibody, the cells may then be rinsed in buffer to remove any unbound antibody. Cells expressing SSEA3, SSEA4, TRA1-60, TRA1-81, Thy-1 or c-kit cell surface antigen can be cell sorted by fluorescence-activated cell sorting using, for example, a Becton Dickinson FACStar flow cytometer. The placental stem cells of this invention may be differentiated directly without additional enrichment and/or purification steps.

d) Methods of Differentiating Placental Stem Cells and Differentiated Cell Types

The placental stem cells may be contacted with various growth factors (termed differentiation factors) that influence differentiation of such stem cells into particular cell types such as hepatocytes, pancreatic cells, vascular endothelial cells, cardiomyocytes and neural cells.

The term “hepatocytes” as used herein refers to cells that have characteristics of epithelial cells obtained from liver. Hepatocytes are cells that express markers such as asialoglycoprotein receptor (ASGR), alpha-1-antitrypsin (A1AT), albumin, hepatocyte nuclear factors (HNF1 and HNF4) and cytochrome P450 (CYP) genes (1A1, 1A2, 2A6, 2B6, 2C8, 2C9, 2C18, 2D6, 3A4, 3A5, 3A7, 4A11). Other markers of interest for hepatocytes include α1-antitrypsin, glucose-6-phosphatase, transferrin, cytokeratin 7 (CK7), γ-glutamyl transferase; hepatocyte nuclear factors (HNF 1β, HNF 3α, HNF-4α), transthyretin, cystic fibrosis transmembrane conductance regulator (CFTR), glucokinase, insulin growth factors (IGF) 1 and 2, IGF-1 receptor, insulin receptor, leptin, apolipoproteins (apoE, apoAII, apoB, apoCIII, apoCII), aldolase B, phenylalanine hydroxylase, L-type fatty acid binding protein, transferrin, retinol binding protein, erythropoietin (EPO), and clotting factors, such as Factor V, VII, VIII, IX and X.

Placental stem cells may be differentiated into hepatocytes by culturing the cells in a media containing at least one hepatocyte differentiation factor. Examples of hepatocyte differentiation factors include epidermal growth factor EGF (0.1-100 ng/ml); dexamethasone (0.1-100 μM); hepatocyte growth factor HGF (0.1-100 ng/ml); insulin (0.1-100 μg/ml), transferrin (0.1-100 μg/ml), selenium (0.1-100 ng/ml, ethanolamine (0.1-100 μg/ml), phenobarbital (1 mM), Type-I collagen. A preferred medium for differentiation of stem cells into hepatocytes includes 10 ng/ml EGF, 0.1 μM dexamethasone, 10 μg/ml insulin, 5.5 μg/ml transferrin, 6.7 ng/ml selenium, and 2 μg/ml ethanolamine. Particularly preferred hepatocytes were deposited with American Type Culture Collection on ______ and have been assigned ATCC accession number ______.

As used herein, the term “pancreatic cell” is used to refer to cells that produce glucagon, somatostatin, pancreatic polypeptide (PP) and/or insulin. Preferred pancreatic cells are positive for pancreatic cell specific markers, such as homeobox transcription factor Nkx-2.2, glucagon, paired box gene 6 (Pax6), pancreatic duodenal homeobox 1 (Pdx1), and insulin.

Placental stem cells can be differentiated into pancreatic cells by culturing the cells in media supplemented with at least one pancreatic cell differentiation factor, such as nicotinamide (10 mM), dexamethasone (0.1 μM), insulin-transferrin-selenium (ITS) or Matrigel™. Particularly preferred pancreatic cells were deposited with American Type Culture Collection on ______ and have been assigned ATCC accession number ______.

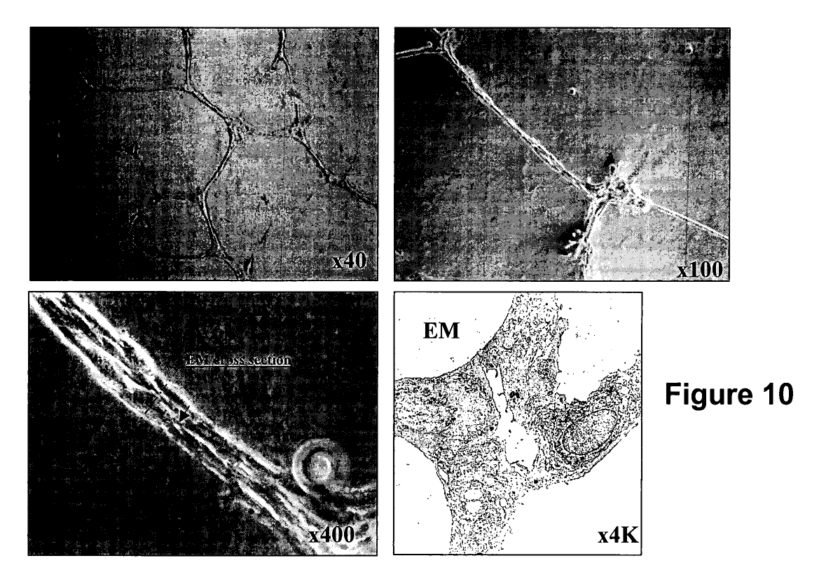

As used herein, the term “vascular endothelial cell” refers to an endothelial cell that exhibits essential physiological functions characteristic of vascular endothelial cells including modulation of vasoreactivity and provision of a semi-permeable barrier to plasma fluid and protein. Phenotypically, vascular endothelial cells may appear similar to the cells shown in FIG. 10. Preferred vascular endothelial cell express a marker including but not limited to vascular cell adhesion molecule-1 (VCAM-1), FMS-like tyrosine kinase 1 (FLT-1, also known as vascular endothelial growth factor (VEGF) receptor-1) and RGD (arginine-glycine-aspartic acid)-dependent integrins, including the vitronectin receptor (alphaᵥbeta₃ or .alphaᵥbeta₅), the collagen Types I and IV receptor (alpha₁beta₁), the laminin receptor (alpha₂beta₁), the fibronectin/laminin/collagen receptor (alpha₃beta₁) and the fibronectin receptor (Davis et al., J. Cell. Biochem. 51:206-218 (1993)).

Placental stem cells can be differentiated into vascular endothelial cells by culturing the cells in a media supplemented with at least one vascular endothelial cells differentiation factor, such as Matrigel™, vascular endothelial growth factor (VEGF), fibroblast growth factor-1 (FGF-I), fibroblast growth factor-2 (FGF-2), platelet-derived endothelial cell growth factor (PD-ECGF), and platelet-derived growth factor (PDGF). Particularly preferred vascular endothelial cells were deposited with American Type Culture Collection on ______ and have been assigned ATCC accession number ______.

The term “cardiomyocyte” as used herein refers to a cardiac muscle cell that may spontaneously beat or may exhibit calcium transients (flux in intracellular calcium concentrations measurable by calcium imaging). Preferred cardiomyoctes express at least one cardiomyocyte specific marker such as cardiac transcription factor-4 (GATA-4), cardiogenic homeodomain factor Nkx 2.5, atrial myosin light chain type 2 (MLC-2A), ventricular myosin light chain type 2 (MLC-2V), human atrial natriuretic peptide (hANP), cardiac troponin T (cTnT), cardiac troponin I (cTnI), alpha-actinin, sarcomeric myosin heavy chain (MHC), N-cadherin, beta1-adrenoceptor (beta1-AR), the myocyte enhancer factor-2 (MEF-2) family of transcription factors, creatine kinase MB (CK-MB), or myoglobin.

Placental stem cells can be differentiated into cardiomyocytes by culturing the cells in a media supplemented with at least one cardiomyocyte differentiation factor, such as L-ascorbic acid 2-phosphate (1 mM), 5-aza-deoxy-cytidine (1 to 10 μM), forskolin (10 μM), growth factors including epidermal growth factor (EGF), fibroblast growth factor (FGF) (preferably basic fibroblast growth factor (bFGF), fibroblast growth factor-4 (FGF-4), fibroblast growth factor-8 (FGF-8), atrial natiuretic factor, transforming growth factor-beta (TGF-beta), activin (A and B), bone morphogenic protein (BMP-4), Leukemia inhibitory factor (LIF), platelet derived growth factor-beta (PDGF-beta), transforming growth factor-alpha (TGF-alpha) at a protein concentration of 1-100 ng/ml, insulin like growth factor-II (IGF-II) (1-100 nM), and insulin (1-100 nM). Particularly preferred cardiomyocytes were deposited with American Type Culture Collection on ______ and have been assigned ATCC accession number ______.

As used herein “neural cells” refer to cells that exhibit essential functions of neurons, and glial cells (astrocytes and oligodendrocytes). Preferred neural cells express at least one neural cell specific marker such as nestin, neuron specific enolase (NSE), neurofilament-M (NF-M), beta-tubulin, C-type natriuretic peptide (CNP), glutamic acid decarboxylase (GAD), tau, microtubule-associated protein 2a and b (MAP2), neurogenin, neuron specific nuclear protein (Neu N), a Hu protein (A, B, C, D), glial fibrillary acid protein (GFAP), oligodendrocyte marker 4 (O4), galactocerebroside (GalC), or myelin basic protein (MBP).

Placental stem cells can be differentiated into neural cells by culturing the cells in media that include a neural cell differentiation factor such as all trans retinoic acid, epidermal growth factor (EGF) (0.1-100 ng/ml), dexamethasone (0.1-100 μM), hepatocyte growth factor (HGF) (0.1-100 ng/ml), insulin (0.1-100 μg/ml)-transferrin (0.1-100 μg/ml)-selenium (0.1-100 ng/ml) (ITS), ethanolamine (0.1-100 μg/ml) and, in particular, with fibroblast growth factor 4 (FGF-4), preferably in the range of 10 ng/ml, nerve growth factor (NGF), transforming growth factor-alpha (TGF-alpha), brain-derived neurotrophic factor (BDNF), glial-derived neurotrophic factor (GDNF), acidic fibroblast growth factor (aFGF of FGF-1), basic fibroblast growth factor (bFGF or FGF2), leukemia inhibitory factor (LIF), platelet-derived growth factor (PDGF), ciliary neurotrophic factor (CNTF), neurotrophin-3, neurotrophin-4, amphiregulin, and Notch antagonists. Particularly preferred neural cells were deposited with American Type Culture Collection on ______ and have been assigned ATCC accession number ______.

Differentiated cells derived from placental stem cells may be detected and/or enriched by the detection of tissue-specific markers by immunological techniques, such as flow immunocytochemistry for cell-surface markers, immunohistochemistry (for example, of fixed cells or tissue sections) for intracellular or cell-surface markers, Western blot analysis of cellular extracts, and enzyme-linked immunoassay, for cellular extracts or products secreted into the medium. The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard amplification methods.

Alternatively, differentiated cells may be detected using selection markers. For example, placental stem cells can be stably transfected with a marker that is under the control of a tissue-specific regulatory region as an example, such that during differentiation, the marker is selectively expressed in the specific cells, thereby allowing selection of the specific cells relative to the cells that do not express the marker. The marker can be, e.g., a cell surface protein or other detectable marker, or a marker that can make cells resistant to conditions in which they die in the absence of the marker, such as an antibiotic resistance gene (see e.g., in U.S. Pat. No. 6,015,671).

3) Therapeutic Uses of Placental Stem Cells and Differentiated Cells

Compositions comprising placental stem cells or cells differentiated therefrom may be administered to a subject to provide various cellular or tissue functions. As used herein “subject” may mean either a human or non-human animal.

Such compositions may be formulated in any conventional manner using one or more physiologically acceptable carriers optionally comprising excipients and auxiliaries. Proper formulation is dependent upon the route of administration chosen. The compositions may be packaged with written instructions for use of the cells in tissue regeneration, or restoring a therapeutically important metabolic function. Placental stem cells may also be administered to the recipient in one or more physiologically acceptable carriers. Carriers for these cells may include, but are not limited to, solutions of phosphate buffered saline (PBS) or lactated Ringer's solution containing a mixture of salts in physiologic concentrations.

One of skill in the art may readily determine the appropriate concentration of cells for a particular purpose. A preferred dose is in the range of about 0.25-1.0 times 10⁶ cells.

Placental stem cells or differentiated cells can be administered by injection into a target site of a subject, preferably via a delivery device, such as a tube, e.g., catheter. In a preferred embodiment, the tube additionally contains a needle, e.g., a syringe, through which the cells can be introduced into the subject at a desired location. Specific, non-limiting examples of administering cells to subjects may also include administration by subcutaneous injection, intramuscular injection, or intravenous injection. If administration is intravenous, an injectible liquid suspension of cells can be prepared and administered by a continuous drip or as a bolus.

Cells may also be inserted into a delivery device, e.g., a syringe, in different forms. For example, the cells can be suspended in a solution contained in such a delivery device. As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating placental stem cells or differentiated cells as described herein, in a pharmaceutically acceptable carrier or diluent and, as required, other ingredients enumerated above, followed by filter sterilization.

The cells may be administered systemically (for example intravenously) or locally (for example directly into a myocardial defect under echocardiogram guidance, or by direct application under visualization during surgery). For such injections, the cells may be in an injectible liquid suspension preparation or in a biocompatible medium which is injectible in liquid form and becomes semi-solid at the site of damaged tissue. A conventional intra-cardiac syringe or a controllable endoscopic delivery device can be used so long as the needle lumen or bore is of sufficient diameter (e.g. 30 gauge or larger) that shear forces will not damage the cells being delivered.

Cells may be administered in a manner that permits them to graft to the intended tissue site and reconstitute or regenerate the functionally deficient area. See Example 7. Both types of cells can be used in therapy by direct administration, or as part of a bioassist device that provides temporary or permanent organ function.

Alternatively, placental stem cells or differentiated cells may be transplanted into the recipient where the cells will proliferate and differentiate to form new cells and tissues thereby providing the physiological processes normally provided by that tissue. The term “transplanted” as used herein refers to either transferring the cells that are embedded in a support matrix or transferring tissues formed by differentiated cells derived from placental stem cells to a subject in need thereof. As used herein, the term “tissue” refers to an aggregation of similarly specialized cells united in the performance of a particular function. Tissue is intended to encompass all types of biological tissue including both hard and soft tissue. Soft tissue refers to tissues that connect, support, or surround other structures and organs of the body. Soft tissue includes muscles, tendons (bands of fiber that connect muscles to bones), fibrous tissues, fat, blood vessels, nerves, and synovial tissues (tissues around joints). Hard tissue includes connective tissue (e.g., hard forms such as osseous tissue or bone) as well as other muscular or skeletal tissue.

Support matrices into which the placental stem cells can be incorporated or embedded include matrices which are recipient-compatible and which degrade into products which are not harmful to the recipient. These matrices provide support and protection for placental stem cells and differentiated cells in vivo and are, therefore, the preferred form in which such cells are transplanted into the recipient subjects.

Natural and/or synthetic biodegradable matrices are examples of such matrices. Natural biodegradable matrices include plasma clots, e.g., derived from a mammal, collagen, fibronectin, and laminin matrices. Suitable synthetic material for a cell transplantation matrix must be biocompatible to preclude migration and immunological complications, and should be able to support extensive cell growth and differentiated cell function. It must also be resorbable, allowing for a completely natural tissue replacement. The matrix should be configurable into a variety of shapes and should have sufficient strength to prevent collapse upon implantation. Recent studies indicate that the biodegradable polyester polymers made of polyglycolic acid fulfill all of these criteria, as described by Vacanti, et al. J. Ped. Surg. 23:3-9 (1988); Cima, et al. Biotechnol. Bioeng. 38:145 (1991); Vacanti, et al. Plast. Reconstr. Surg. 88:753-9 (1991). Other synthetic biodegradable support matrices include synthetic polymers such as polyanhydrides, polyorthoesters, and polylactic acid. Further examples of synthetic polymers and methods of incorporating or embedding cells into these matrices are also known in the art. See e.g., U.S. Pat. Nos. 4,298,002 and 5,308,701.

Attachment of the cells to the polymer may be enhanced by coating the polymers with compounds such as basement membrane components, agar, agarose, gelatin, gum arabic, collagens types I, II, III, IV and V, fibronectin, laminin, glycosaminoglycans, mixtures thereof, and other materials known to those skilled in the art of cell culture. All polymers for use in the matrix must meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy, with respect to toxicology by initial screening tests involving Ames assays and in vitro teratogenicity assays, and implantation studies in animals for immunogenicity, inflammation, release and degradation studies.

One of the advantages of a biodegradable polymeric matrix is that angiogenic and other bioactive compounds can be incorporated directly into the support matrix so that they are slowly released as the support matrix degrades in vivo. As the cell-polymer structure is vascularized and the structure degrades, placental stem cells may differentiate according to their inherent characteristics. Factors, including nutrients, growth factors, inducers of differentiation or de-differentiation (i.e., causing differentiated cells to lose characteristics of differentiation and acquire characteristics such as proliferation and more general function), products of secretion, immunomodulators, inhibitors of inflammation, regression factors, biologically active compounds which enhance or allow ingrowth of the lymphatic network or nerve fibers, hyaluronic acid, and drugs, which are known to those skilled in the art and commercially available with instructions as to what constitutes an effective amount, from suppliers such as Collaborative Research, Sigma Chemical Co., vascular growth factors such as vascular endothelial growth factor (VEGF), epidermal growth factor (EGF), and heparin binding epidermal growth factor like growth factor (HB-EGF), could be incorporated into the matrix or provided in conjunction with the matrix. Similarly, polymers containing peptides such as the attachment peptide RGD (Arg-Gly-Asp) can be synthesized for use in forming matrices (see e.g U.S. Pat. Nos. 4,988,621, 4,792,525, 5,965,997, 4,879,237 and 4,789,734).

In another example, the cells may be transplanted in a gel matrix (such as Gelfoam from Upjohn Company) which polymerizes to form a substrate in which the placental stem cells or differentiated cells can grow. A variety of encapsulation technologies have been developed (e.g. Lacy et al., Science 254:1782-84 (1991); Sullivan et al., Science 252:718-712 (1991); WO 91/10470; WO 91/10425; U.S. Pat. No. 5,837,234; U.S. Pat. No. 5,011,472; U.S. Pat. No. 4,892,538). During open surgical procedures, involving direct physical access to the damaged tissue and/or organ, all of the described forms of undifferentiated placental stem cells or differentiated placental stem cell delivery preparations are available options. These cells can be repeatedly transplanted at intervals until a desired therapeutic effect is achieved.

The present invention also relates to the use of placental stem cells in three dimensional cell and tissue culture systems to form structures analogous to tissue counterparts in vivo. The resulting tissue will survive for prolonged periods of time, and perform tissue-specific functions following transplantation into the recipient host. Methods for producing such structures are described in U.S. Pat. No. 5,624,840 and 6,428,802, which are incorporated herein in their entireties.

The three-dimensional matrices to be used are structural matrices that provide a scaffold for the cells, to guide the process of tissue formation. Scaffolds can take forms ranging from fibers, gels, fabrics, sponge-like sheets, and complex 3-D structures with pores and channels fabricated using complex Solid Free Form Fabrication (SFFF) approaches. Cells cultured on a three-dimensional matrix will grow in multiple layers to develop organotypic structures occurring in three dimensions such as ducts, plates, and spaces between plates that resemble sinusoidal areas, thereby forming new liver tissue. Thus, in preferred aspects, the present invention provides a three-dimensional framework, multi-layer cell and tissue culture system. As used herein, “three-dimensional framework” refers to a three-dimensional scaffold composed of any material and/or shape that (a) allows cells to attach to it (or can be modified to allow cells to attach to it); and (b) allows cells to grow in more than one layer. The structure of the framework can include a mesh, a sponge or can be formed from a hydrogel.

Examples of such frameworks include a three-dimensional stromal tissue or living stromal matrix which has been inoculated with stromal cells that are grown on a three dimensional support. The extracellular matrix proteins elaborated by the stromal cells are deposited onto the framework, thus forming a living stromal tissue. The living stromal tissue can support the growth of placental stem cells or differentiated cells later inoculated to form the three-dimensional cell culture. Examples of other three dimensional frameworks are described in U.S. Pat. No. 6,372,494.

The design and construction of the scaffolding to form a three-dimensional matrix is of primary importance. The matrix should be a pliable, non-toxic, injectable porous template for vascular ingrowth. The pores should allow vascular ingrowth. These are generally interconnected pores in the range of between approximately 100 and 300 microns, i.e., having an interstitial spacing between 100 and 300 microns, although larger openings can be used. The matrix should be shaped to maximize surface area, to allow adequate diffusion of nutrients, gases and growth factors to the cells on the interior of the matrix and to allow the ingrowth of new blood vessels and connective tissue. At the present time, a porous structure that is relatively resistant to compression is preferred, although it has been demonstrated that even if one or two of the typically six sides of the matrix are compressed, that the matrix is still effective to yield tissue growth.

The polymeric matrix may be made flexible or rigid, depending on the desired final form, structure and function. For repair of a defect, for example, a flexible fibrous mat is cut to approximate the entire defect, then fitted to the surgically prepared defect as necessary during implantation. An advantage of using the fibrous matrices is the ease in reshaping and rearranging the structures at the time of implantation.

A sponge-like structure can also be used to create a three-dimensional framework. The structure should be an open cell sponge, one containing voids interconnected with the surface of the structure, to allow adequate surfaces of attachment for sufficient placental stem cells or differentiated cells to form a viable, functional implant.

Placental stem cells and cells differentiated therefrom may also be used to humanize animal organs. Example 7 demonstrates transplantation of human placental stem cells into mouse liver and data showing the differentiation of the cells into human hepatocytes within the mouse liver.

Human placental stem cells may be similarly transplanted into another organ such as pancreas or brain or heart. The animal organ may or may not be depleted of its native cells prior to the transplant. “Humanized” organs of an animal such as a mouse, rat, monkey, pig or dog could be useful for organ transplants into humans with specific diseases.

Humanized animal models may also be used for diagnostic or research purposes relating but not limited to, drug metabolism, toxicology studies or for the production, study, or replication of viral or bacterial organisms. Mice transplanted with human hepatocytes forming chimeric human livers are currently being used for the study of hepatitis viruses (Dandri et al. Hepatol. 33:981-988 (2001), and Mercer et al. Nature Med. 7:927-933 (2001)).

Placental stem cells may be genetically engineered to produce a particular therapeutic protein. As used herein the term “therapeutic protein” includes a wide range of biologically active proteins including, but not limited to, growth factors, enzymes, hormones, cytokines, inhibitors of cytokines, blood clotting factors, peptide growth and differentiation factors. Particular differentiated cells may be engineered with a protein that is normally expressed by the particular cell type. For example, pancreatic cells can be engineered to produce digestive enzymes. Hepatocytes can be engineered to produce the enzyme inhibitor, A1AT, or clotting factors to treat hemophilia. Furthermore, neural cells can be engineered to produce chemical transmitters.

Methods which are well known to those skilled in the art can be used to construct expression vectors containing a nucleic acid encoding the protein of interest linked to appropriate transcriptional/translational control signals. See, for example, the techniques described in Sambrook, et al. Molecular Cloning, A Laboratory Manual, Cold Spring Harbor Laboratory, N.Y. (1992) and Ausebel et al. Current Protocols in Molecular Biology, Greene Publishing Associates & Wiley Interscience, N.Y (1989).

Suitable methods for transferring vector or plasmids into placental stem cells or cells differentiated therefrom include lipid/DNA complexes, such as those described in U.S. Pat. Nos. 5,578,475; 5,627,175; 5,705,308; 5,744,335; 5,976,567; 6,020,202; and 6,051,429. Suitable reagents include lipofectamine, a 3:1 (w/w) liposome formulation of the poly-cationic lipid 2,3-dioleyloxy-N-[2(sperminecarbox-amido)ethyl]-N,N-dimethyl-1-propanaminium trifluoroacetate (DOSPA) (Chemical Abstracts Registry name: N-[2-(2,5-bis[(3-aminopropyl)amino]-1-oxpentyl)amino)ethyl]-N,N-dimethyl-2,3-bis(9-octadecenyloxy)-1-propanamin-trifluoroacetate), and the neutral lipid dioleoyl phosphatidylethanolamine (DOPE) in membrane filtered water. Exemplary is the formulation Lipofectamine 2000™ (available from Gibco/Life Technologies # 11668019). Other reagents include: FuGENE™ 6 Transfection Reagent (a blend of lipids in non-liposomal form and other compounds in 80% ethanol, obtainable from Roche Diagnostics Corp. # 1814443); and LipoTAXI™ transfection reagent (a lipid formulation from Invitrogen Corp., produce the desired biologically active protein. #204110). Transfection of placental stem cells can be performed by electroporation, e.g., as described in Roach and McNeish (Methods in Mol. Biol. 185:1 (2002)). Suitable viral vector systems for producing stem cells with stable genetic alterations may be based on adenoviruses, lentiviruses, retroviruses and other viruses, and may be prepared using commercially available virus components.

Placental stem cells that have been differentiated may be administered or transplanted to a subject to provide various cellular or tissue functions specific to the differentiated cell type. For example, placental stem cells that have been differentiated into hepatocytes can be used in the treatment of liver diseases, such as in artificial liver devices (BAL-bioartificial liver) or for hepatocyte transplant. The term “liver disease” as used herein includes but is not limited to cirrhosis of the liver, metabolic diseases of the liver, such as alpha 1-antitrypsin deficiency and omithine transcarbamylase (OTC), alcohol-induced hepatitis, chronic hepatitis, primary sclerosing cholangitis, alpha 1-antitrypsin deficiency and liver cancer.

Hepatocytes of the invention can be assessed in animal models for ability to repair liver damage. One such example is damage caused by intraperitoneal injection of D-galactosamine (Dabeva et al. Am. J. Pathol. 143:1606 (1993)). Efficacy of treatment can be determined by immunocytochemical staining for liver cell markers, microscopic determination of whether canalicular structures form in growing tissue, and the ability of the treatment to restore synthesis of liver-specific proteins.

Hepatocytes can be grown on a three-dimensional matrix in vitro under conditions effective and for a period of time sufficient to allow proliferation of the cells to form a three-dimensional structure. To form the bio-artificial liver the three-dimensional hepatocyte cell cultures of the invention are grown within a containment vessel containing an input and output outlet for passage of the subject's blood through the containment vessel. The bio-artificial liver further includes a blood input line which is operatively coupled to a conventional peristaltic pump. A blood output line is also included. Input and output lines are connected to appropriate arterial-venous fistulas which are implanted into, for example, the forearm of a subject. In addition, the containment vessel may contain input and output outlets for circulation of appropriate growth medium to the hepatocytes for continuous cell culture within the containment vessel. The use of such bio-artificial livers involves the perfusion of the subject's plasma through the bio-artificial liver. In the perfusion protocol, the subject's blood or plasma is withdrawn and passes into contact with the hepatocyte cell cultures. During such passage, molecules dissolved in the patient's blood, such as bilirubin, are taken up and metabolized by the hepatocyte cultures. In addition, the cultured hepatocytes provide factors normally supplied by liver tissue.

The progress of the recipient receiving such cells or transplants can be determined using assays that include blood tests known as liver function tests. Such liver function tests include assays for alkaline phosphates, alanine transaminase, aspartate transaminase and bilirubin circulating levels of liver derived clotting factors and determination of clotting times. In addition, recipients can be examined for presence or disappearance of features normally associated with liver disease such as, for example, jaundice, anemia, leukopenia, thrombocytopenia, increased heart rate, and high levels of insulin. Additionally, assays specific for measuring deficiencies in particular metabolic disorders may also be used. Further, imaging tests such as ultrasound, computer assisted tomography (CAT) and magnetic resonance (MR) may be used to assay for liver function.

Hepatocytes derived from placental stem cells may be used in assays to detect the activity of specific metabolic pathways. The cells may have a positive response to dibenzylfluorescein (DBF), have the ability to metabolize certain drugs, e.g., dextromethorphan and coumarin; have drug efflux pump activities (e.g., P glycoprotein activity); upregulation of CYP activity by phenobarbital, as measured, e.g., with the pentoxyresorufin (PROD) assay, which is seen only in hepatocytes and not in other cells (see, e.g., Schwartz et al. J. Clin. Invest. 109:1291 (2002)); take up LDL, e.g., Dil-acil-LDL (see, e.g., Schwartz et al., supra); store glycogen, as determined, e.g., by using a periodic acid-Schiff (PAS) staining of the cells (see, e.g., Schwartz et al., supra); produce urea and albumin (see, e.g., Schwartz et al., supra); and present evidence of glucose-6-phosphatase activity.

Pancreatic cells derived from placental stem cells can be used therapeutically for treatment of various diseases associated with insufficient functioning of the pancreas. As used herein, the term “pancreatic disease” may include but is not limited to pancreatic cancer, insulin-deficiency disorder such as Insulin-dependent (Type 1) diabetes mellitus (IDDM) and Non-insulin-dependent (Type 2) diabetes mellitus (NIDDM), hepatitis C infection, exocrine and endocrine pancreatic diseases.

Example 9 shows cultured placental stem cells that express pancreatic islet cell markers, in particular, insulin. These cells, therefore, may secrete or be induced to secrete insulin for use towards the treatment of diabetes.

The placental stem cells can be used to produce populations of differentiated pancreatic cells for repair subsequent to partial pancreatectomy, e.g., excision of a portion of the pancreas. Likewise, such cell populations can be used to regenerate or replace pancreatic tissue loss due to, pancreatolysis, e.g., destruction of pancreatic tissue, such as pancreatitis, e.g., a condition due to autolysis of pancreatic tissue caused by escape of enzymes into the substance. Pancreatic cells may be transplanted into the pancreas or to ectopic sites, such as, but not limited to the liver, kidney or at or near the intestines.

Methods of administration include encapsulating differentiated β islet cells producing insulin in implantable hollow fibers. Such fibers can be pre-spun and subsequently loaded with the differentiated β islet cells of the invention (see U.S. Pat. No. 4,892,538; U.S. Pat. No. 5,106,627; Hoffman et al. Expt. Neurobiol. 110:39-44 (1990); Jaeger et al. Prog. Brain Res. 82:41-46 (1990); and Aebischer et al. J. Biomech. Eng. 113:178-183 (1991)), or can be co-extruded with a polymer which acts to form a polymeric coat about the β islet cells (U.S. Pat. No. 4,391,909; U.S. Pat. No. 4,353,888; Sugamori et al. Trans. Am. Artif. Intern. Organs 35:791-799 (1989); Sefton et al. Biotehnol. Bioeng. 29:1135-1143 (1987); and Aebischer et al. Biomaterials 12:50-55 (1991)).

The present invention also provides for administration of neural cells derived from placental stem cells for treatment of neurological disease. The term “neurological disease” refers to a disease or condition associated with any defects in the entire integrated system of nervous tissue in the body: the cerebral cortex, cerebellum, thalamus, hypothalamus, midbrain, pons, medulla, brainstem, spinal cord, basal ganglia and peripheral nervous system. Examples include but are not limited to: Parkinson's disease, Huntington's disease, Multiple Sclerosis, Alzhemier's disease, amylotrophic lateral sclerosis (ALS or Lou Gerhig's disease), Muscular dystrophy, choreic syndrome, dystonic syndrome, stroke, and paralysis.

The placental stem cells may be used in in vitro priming procedures that result in neural stem cells becoming neurons when grafted into non-neurogenic or neurogenic areas of the CNS. Transplanted cells further differentiate by acquiring cholinergic, glutamatergic and/or GABAergic phenotypes in a region-specific manner. For example, when transplanted into medial septum or spinal cord, they preferentially differentiate into cholinergic neurons; when transplanted into frontal cortex they preferentially differentiate into glutamatergic neurons; and when transplanted into hippocampus they preferentially differentiate into GABAergic neurons. Neurons “preferentially differentiate” into neurons of a specific phenotype when at least 50% of the neurons are of a specific phenotype. These neurons can be used to replace the neurons lost or damaged in neurodegenerative disease, including, but not limited to AD and ALS, or neurotrauma, including, but not limited to, spinal cord injury, head injury and stroke-related dementia.

In an exemplary embodiment, a pharmaceutical composition comprising an effective amount of the vascular endothelial cells may be used to treat a subject with a vascular disease. As used herein, “vascular disease” refers to a disease of the human vascular system. Examples include peripheral arterial disease, abdominal aortic aneurysm, carotid disease, and venous disease.

The placental stem cells can be used to produce vascular endothelial cells that may be used in methods for remodeling tissue or replacing a scar tissue in a subject. Vascular endothelial cells may also be used to repair vascular damage.

The present invention also provides for cardiomyocytes derived from placental stem cells which may be used therapeutically for treatment of various diseases associated with cardiac dysfunction. The term “cardiac disease” or “cardiac dysfunction” as used herein refers to diseases that result from any impairment in the heart's pumping function. This includes, for example, impairments in contractility, impairments in ability to relax (sometimes referred to as diastolic dysfunction), abnormal or improper functioning of the heart's valves, diseases of the heart muscle (sometimes referred to as cardiomyopathy), diseases such as angina and myocardial ischemia and infarction characterized by inadequate blood supply to the heart muscle, infiltrative diseases such as amyloidosis and hemochromatosis, global or regional hypertrophy (such as may occur in some kinds of cardiomyopathy or systemic hypertension), and abnormal communications between chambers of the heart (for example, atrial septal defect). For further discussion, see Braunwald, Heart Disease: a Textbook of Cardiovascular Medicine, 5th edition, W B Saunders Company, Philadelphia Pa. (1997) (hereinafter Braunwald). The term “cardiomyopathy” refers to any disease or dysfunction of the myocardium (heart muscle) in which the heart is abnormally enlarged, thickened and/or stiffened. As a result, the heart muscle's ability to pump blood is usually weakened. The disease or disorder can be, for example, inflammatory, metabolic, toxic, infiltrative, fibroplastic, hematological, genetic, or unknown in origin. There are two general types of cardiomyopathies: ischemic (resulting from a lack of oxygen) and nonischemic. Other diseases include congenital heart disease which is a heart-related problem that is present since birth and often as the heart is forming even before birth or diseases that result from myocardial injury which involves damage to the muscle or the myocardium in the wall of the heart as a result of disease or trauma. Myocardial injury can be attributed to many things such as, but not limited to, cardiomyopathy, myocardial infarction, or congenital heart disease.

The placental stem cells and/or differentiated cardiomyocytes may be administered and/or transplanted to a subject suffering from a cardiac disease in any fashion as previously discussed.

Methods are also provided for screening agents that affect cardiomyocyte differentiation or function. According to one method, a population of cardiomyocytes may be produced as described herein, a population of cells is contacted with an agent of interest, and the effect of the agent on the cell population is then assayed. For example, the effect on differentiation, survival, proliferation, or function of the cells may then be assessed. Such screening assays may involve the measurement of calcium transients. In one embodiment calcium imaging is used to measure calcium transients. For example, ratiometric dyes, such as fura-2, fluo-3, or fluo-4 are used to measure intracelluar calcium concentration. The relative calcium levels in a population of cells treated with a ratiometric dye can be visualized using a fluorescent microscope or a confocal microscope. In other embodiments, the membrane potential across the cell membrane is monitored to assess calcium transients. For example, a voltage clamp may be used. In this method, an intracellular microelectrode is inserted into the cardiomyocyte. In one embodiment, calcium transients can be seen before observable contractions of the cardiomyocytes. In other embodiments calcium transients are seen either during, or after, observable contractions of cardiomyocytes. In another embodiment the cells are cultured in the presence of conditions wherein the cells do not beat, such as in the presence of a calcium chelator (e.g. EDTA or EGTA) and the calcium transients are measured.

Any other method known to one of skill in the art may be utilized to assess cardiac function. In one embodiment the beating rate of a cardiomyocyte may also be assayed to identify agents that increase or decrease beating. One method for assessing the beating rate is to observe beating under a microscope. Agents that can be screened in this manner include inotropic drugs, such as sympathomimetic agents.

In another embodiment, placental stem cells, and their derivatives, can be used to screen various compounds to determine the effect of the compound on cellular growth, proliferation or differentiation of the cells. Methods of measuring cell proliferation are well known in the art and most commonly include determining DNA synthesis characteristic of cell replication. There are numerous methods in the art for measuring DNA synthesis, any of which may be used according to the invention. For example, DNA synthesis may be determined using a radioactive label (³H-thymidine) or labeled nucleotide analogues (BrdU) for detection by immunofluorescence. The efficacy of the compound can be assessed by generating dose response curves from data obtained using various concentrations of the compound. A control assay can also be performed to provide a baseline for comparison. Identification of the placental stem cell population(s) amplified in response to a given test agent can be carried out according to such phenotyping as described above.

In order to assess the effect of a test agent on placental stem cell differentiation or function, the agent may be contacted with the placental stem cells and differentiation assessed using any means known to one of skill in the art. For example, the morphology can be examined using electron microscopy. Immunohistochemical or immunofluorescence techniques may also be used to assess differentiation. Differentiation may be further assessed by analyzing expression of specific mRNA molecules expressed in specific differentiated cells. Suitable assay systems include, but are not limited to RT-PCR, in situ hybridization, Northern analysis, or RNase protection assays. In a further embodiment the levels of polypeptides expressed in differentiated cell types are assayed. Specific, non-limiting examples of polypeptide assays include Western blot analysis, ELISA assay, or immunofluorescence.

Differentiated cells may be used to test whether test agents such as lead drug compounds have a negative biological effect on the cells. For example, the hepatocyte cell preparation may be incubated in the presence or absence of a test compound for a time sufficient to determine whether the compound may be cytotoxic to cells. Differentiated cells can be incubated with various concentrations of a test compound. In an illustrative embodiment, differentiated cells are plated in the wells of a multi-well plate to which different concentrations of the test compound are added, e.g., 0 μM; 0.01 μM; 0.1 μM; 1 μM; 10 μM; 100 μM; 1 mM; 10 mM and 100 mM. Cells can be incubated for various times, e.g., 1 minute, 10 minutes, 1 hour, 2 hours, 5 hours, 10 hours, 24 hours, 36 hours or more.

The biological effect that is measured can be triggering of cell death (i.e., cytotoxicity or hepatotoxicity); a cytostatic effect; or a transforming effect on the cell, as determined, e.g., by an effect on the genotype or phenotype of the cells. The cytotoxicity on cells can be determined, e.g., by incubating the cells with a vital stain, such as trypan blue. Such screening assays can easily be adapted to high throughput screening assays. Differentiated cells derived from placental stem cells of the invention can also be used for metabolic profiling. In one embodiment, cells or a fraction thereof, e.g., a microsome fraction, are contacted with a test agent, potentially at different concentrations and for different times, the media is collected and analyzed to detect metabolized forms of the test agent. Optionally, a control molecule, such as bufuralol is also used. Metabolic profiling can be used, e.g., to determine whether a subject metabolizes a particular drug and if so, how the drug is metabolized.

Exemplifcations

The invention, having been generally described, may be more readily understood by reference to the following examples, which are included merely for purposes of illustration of certain aspects and embodiments of the present invention, and are not intended to limit the invention in any way.

### Example 1

### Isolation and Characterization of Cells Derived from Placental Tissues

The populations of placental cells were isolated from various sections of the placenta. Placental cells were isolated from the amniotic membrane which is easily peeled off of the placental body. The amniotic membrane contains amniotic epithelial cells and a supportive stromal layer which contains mesenchymal cells, or fibroblastic cells as well as other cell types. The amniotic membrane was peeled off of the placenta and was trypsinized to release amniotic epithelial cells. Cells which are derived from the tissue which remains following trypsinization are labeled amniotic fibroblasts (AMF). At this point in the research this fraction is more operationally defined by the mechanism by which cells are released and the tissue from which the cells are derived rather than by histochemically defined cell types. Although the exact cell types in this stromal layer are not fully characterized and defined, to simplify the wording and for the purposes of this application we will call them amniotic fibroblasts (AMF) with full understanding that cell types other than fibroblasts are most likely contained in what we call the AMF fraction.

The amnion layer was peeled off and the remaining placental membrane was digested with collagenase. The cells derived from the remaining tissue was labeled RM. Cells of each fraction (amniotic epithelial cells, amniotic fibroblasts, remaining placental tissue) were plated on plastic culture dishes in basal plating media. At 20 hrs following plating, the cultures were examined. Some cells were attached to the culture dish, referred to as the “adherent fraction”. The remaining cells which did not adhere to the plastic were collected and are represented as “non-adherent fraction”. As stem cells in certain tissues seem to reside in the nonadherent fractions, it is significant that cells with stem cell markers can be found in each of these fractions from placenta. It is not known whether the cells with stem cell characteristics from each fraction are identical. Total cellular RNA was collected from the adherent and non-adherent cells from each of the placental fractions.

Total RNA was extracted with RNAWIZ (Ambion). RT-PCR was performed with Super Script One-step RT-PCR system (GIBCO, 10928-018) with SOX2 and Oct-4 specific primers. RT-PCR with β-actin specific primers was also performed as an internal control.

Results

Both adherent and non-adherent fractions of the amniotic epithelial (AE) cells, the non-adherent fraction of the amniotic fibroblasts (AMF), and adherent fraction of remaining membrane (RM) contain cells express SOX2 and Oct-4. Amniotic epithelial (AE) cells from both adherent and non adherent fractions strongly express SOX2 and Oct-4, while other fractions express primarily SOX2. These different expression patterns of two independent stem cell marker genes indicate that different types of stem cells can be isolated from those fractions. Since neuro-stem cells express SOX2, the results here suggest that the amniotic epithelial fraction as well as the amniotic fibroblast fractions of the placenta contain neuro-stem cells. These results indicate that ES-like cells exist in the fetal side of the placental tissue and can be easily isolated, cultured and identified.

### Example 2

### Expression of Stem Cells Markers, Epithelial Cell Markers and Hepatocyte Markers in Cultured Placental Stem Cells

A human placenta was obtained from an uncomplicated elective caesarean section. The whole placenta was placed in a sterilized 1000 ml cup and washed with Hank's Balanced Salt Solution (HBSS) containing penicillin G (100 U/ml), streptomycin (100 μg/ml), and amphotericin B (0.25 μg/ml). The umbilical cord was cut and the whole placenta was cut in half at the point of attachment of the umbilical cord. The amnion layer was peeled from the underlying chorion layer of the placenta by gentle stripping with a sterile scalpel, starting from the cut edge (middle of the placental body) and working outward. The amnion was washed with HBSS (without antibiotics) and rinsed with 0.05% Trypsin-EDTA. 0.05% Trypsin-EDTA was added to approximately twice the volume of the tissue in a 50 cc Falcon tube and incubated at 37° C. for 20 min on shaker in a 5% CO₂ incubator. The tissue is transferred to a new tube with 0.05% Trypsin-EDTA. Media was added to remaining supernatant in the tube to stop trypsinization and centrifuged at 800 rpm for 10 min at 4° C. The pellet was resuspended in DMEM, 10% FBS, 1 mM Sodium Pyruvate, EGF (10 ng/ml), penicillin G (100 U/ml), streptomycin (100 μg/ml), and amphotericin B (0.25 μg/ml). The trysinization step was repeated up to a total of 3 times. The cells released by each trypsinization were plated separately or mixed in one tube after passing through a 100 μm cell strainer. Cells were plated onto dishes with DMEM, 10% FBS, 2 mM L-glutamine, EGF (10 ng/ml), insulin (10 μg/ml)-transferrin (5.5 μg/ml)-selenium (6.7 ng/ml)-ethanolamine (2 μg/ml) (ITS). The media was changed when the cells adhere on the bottom, approximately 2-4 hrs. Media was changed every two days and the cells were passed (1 in 4) every 5 days or when the cultures reach greater than 80% confluence. Approximately 0.5-2×10⁸ placental stem cells are obtainable from each placenta. Standard culture media (DMEM) was supplemented with 10% FBS, ITS and EGF (10 ng/ml).

Because of potential variability between lines of embryonic stem cells due to isolation techniques and changes in culture, molecular and cellular standards were recently set for the evaluation of human ES cells. In addition to the surface markers such as SSEA-3, SSEA-4, TRA 1-60, TRA 1-81, c-kit and Thy-1, there is consensus agreement that ES lines also express Oct-3/4, SOX-2, Lefty-A, FGF-4, Rex-1, and TDGF-1 (cripto) (Brivanlou, et al. Science 300, 913 (2003)). Cells were collected immediately after isolation and after 24 days of culture (approximately 14 days at confluence) and RNA was isolated. Primers were designed to amplify embryonic stem (ES) cell marker genes as discussed above. For RT-PCT analysis, total RNA was extracted with RNAWIZ (Ambion). RT-PCR was performed with Super Script One-step RT-PCR system (GIBCO, 10928-018) with a human albumin specific primers that were designed to span two-separated exons. RT-PCR with β-actin specific primers was also performed as an internal control. Total RNA extracted from HeLa cells was used as negative control, and RNA from cultured human hepatocytes was used as a positive control.

Cells were also incubated with antibodies to different antigen and analyzed on a flow cytometry analyzer, Beckman-Coulter Epics XL cytometer. Additional cells were analyzed for background fluorescence by incubation with mouse IgG at the same concentration as the highest concentration of antibodies used in this FACs analysis.

Immunohistochemical analysis was also performed at approximately day 5 after placental stem cell isolation. These placental stem cells were trypsinized and replated on collagen-coated cover slips, inserted into 12 well culture plates, and cultured for 2-5 days, then washed 2× with HBSS and fixed with 10% buffered formalin. Prior to cell isolation, a small amount of tissue, approximately 1 cm×1 cm was cut from the placental tissue and fixed in 10% buffered formalin, embedded in paraffin and sectioned. Paraffin-embedded placental tissues were sectioned to 5 μm thickness and placental stem cells, cultured on collagen-coated glass cover slips, were fixed by 10% buffered formalin for immunohistochemical analysis with primary antibodies against AE1/AE3, CK19, CK18, c-kit, Thy-1, A1AT, AFP. Antibody localization was performed using goat anti-mouse immunoglobulins conjugated to biotin. An avidin-biotin peroxidase complex method using DAB as a substrate (Vector) was used to develop the brown˜orange color on positive samples. A hematoxylin counter stain was performed. Immunohistochemical analysis for human HNF-4a was prepared with rabbit anti-human HNF-4 alpha (H171/1:250) antibody (Santa Cruz, sc-8987).

Alkaline phosphatase activity was determined by Vector Red Alkaline phosphatase substrate kit (Vector, SK-5100). Placental stem cells were washed three times with HBSS and fixed by buffered 10% formalin for 2 hr. The red color indicative of alkaline phosphatase positivity was developed per manufacturer's instructions with a 45 min incubation at 37° C.

For Western Blotting experiments, placental stem cells were homogenized in 200 μl RIPA buffer (1% TritonX-100, 150 mM NaCl, 10 mM Tris pH 7.4, 1 mM EDTA, 1 mM EGTA, 0.2 mM sodium vanadate, 0.5% NP-40) and the sample was subjected to electrophoresis on a 10% pre-cast polyacrylamide-SDS gel (Bio-Rad) at 200 V for 30 min, electrically transferred to a nitrocellulose membrane and incubated overnight at 4° C. with mouse anti-human albumin and anti-A1AT antibody.

Results

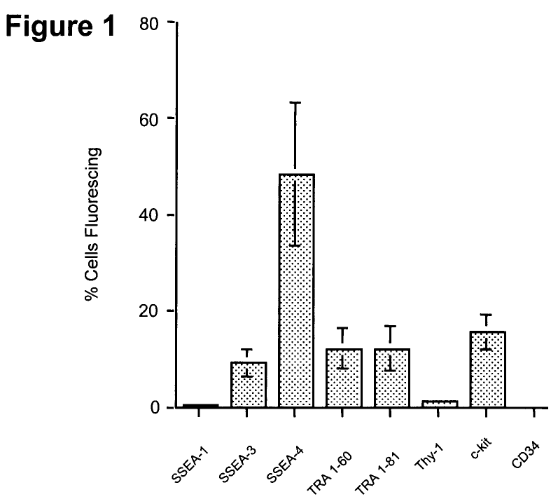

The placental stem cells (PSCs) were analyzed by flow cytometry with antibodies to cell surface markers commonly used to define embryonic stem (ES) cells (Thompson et al. Science 282, 1145 (1998); Reubinoff et al. Nature Biotech. 18, 399 (2000); Draper et al. J. Anat. 200, 249 (2002); Brivanlou et al. Science 300, 913 (2003)). Like human ES cells, PSCs do not express SSEA-1 but do express significant amounts of SSEA-3 (8.79±2.84%), SSEA-4 (43.94±14.8%), TRA 1-60 (9.82±4.31%), and TRA 1-81 (9.91±4.49%) (FIG. 1). Some PSCs react with antibodies to the stem/progenitor cell markers c-kit and Thy-1 (15.39±3.54% and 1.05±0.37%) (Petersen et al. Science 284, 1168 (1999) and Omori et al. Am. J. Pathol. 150, 1179 (1997)) (FIG. 1). While Thy-1 expression is low initially (1.05%), up to 46% of the cells express Thy-1 after 6 days of culture (not shown). Hematopoietic stem cells and rat liver progenitor cells express the Thy-1 antigen (Petersen et al. Science 284:1168-1170(1999); Petersen et al. Hepatology 27:433-445 (1998)). Expression of Thy-1 in placental stem cells indicates that these cells may differentiate to cells of either hematopoietic or hepatic lineage.

The cells do not express the hematopoietic stem cell marker CD34. The absence of CD34 positive cells in this population indicates the isolates are not contaminated with hematopoietic stem cells such as umbilical cord blood cells.

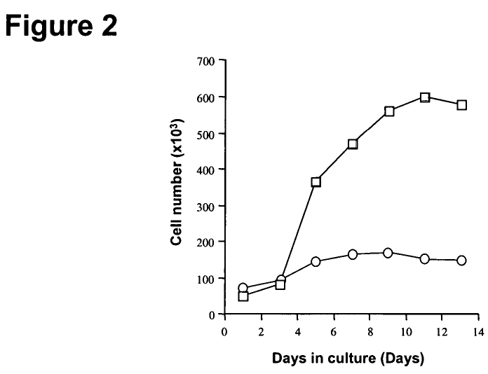

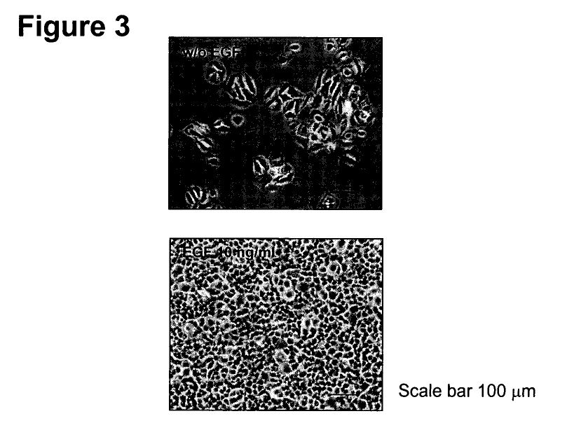

Cells were cultured in the presence or absence of epidermal growth factor (EGF, 10 ng/ml). In the absence of EGF, proliferation ceased and the cells formed giant palm-shaped cells which at later times became multinucleated giant cells reminiscent of the report of trophoblastic differentiation of ES cells (Tanaka et al. Science 282, 2072 (1998)). These senescent cells were not characterized further. In the presence of EGF, PSCs proliferate robustly (FIG. 2) and form confluent monolayers of cobblestone shaped epithelial cells (FIG. 3).

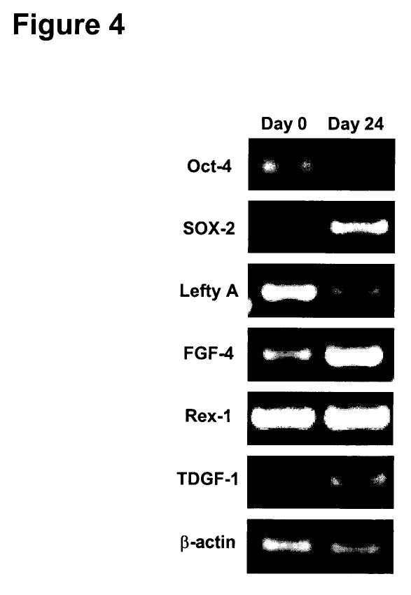

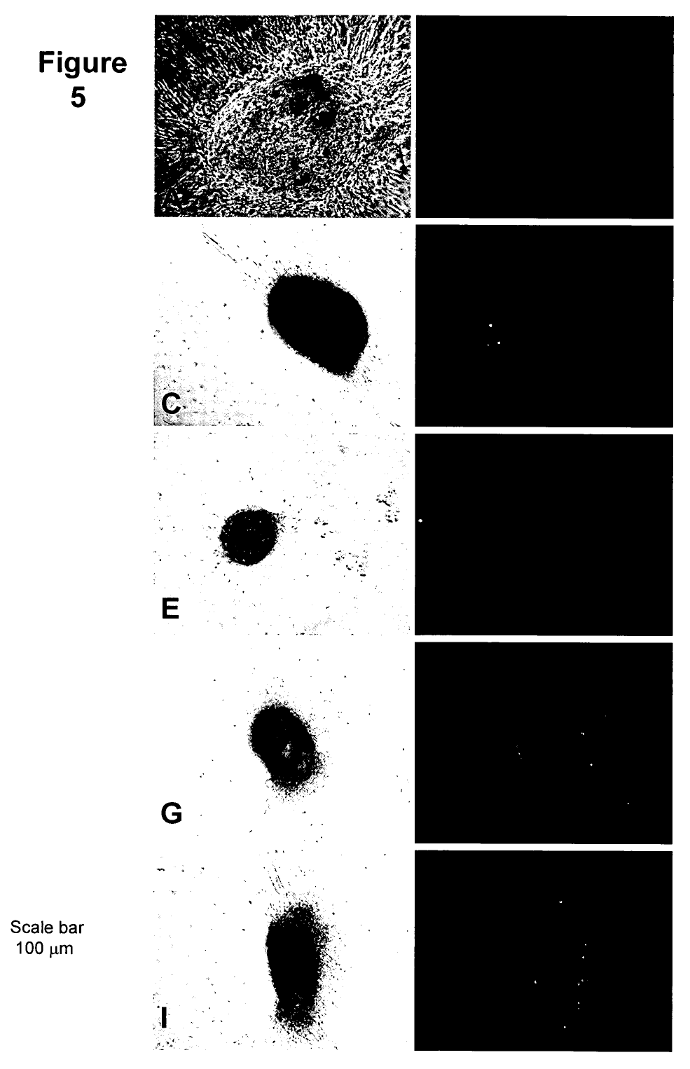

Additionally all of the stem cell marker genes, Oct-3/4, SOX-2, Lefty-A, FGF-4, Rex-1, and TDGF-1 were expressed in the freshly isolated cells as well as in cells cultured for 24 days in the presence of EGF (FIG. 4). The expression of the stem cell markers in the freshly isolated cells was consistent with embryonic stem cells. However, the expression of these markers in confluent cultures was a bit unexpected, as the expression of stem cell markers generally decline when ES cells are maintained at high density, a condition that induces differentiation (Thompson et al. Science 282, 1145, (1998); Reubinoff et al. Nature Biotech. 18, 399 (2000); Draper et al. J. Anat. 200, 249, (2002); Brivanlou, et al. Science 300, 913 (2003)).

Small clusters or spheroids of cells were noticed above the confluent monolayers which are similar in structure to embryoid bodies (EB) described in cultures of ES cells (FIG. 5). Since it was likely that the stem cell markers in the long-term cultures were expressed in these EB-like structures, we examined the expression of the SSEA and TRA antigens in these structures. During expansion with EGF at least two types of cells could be observed; the more predominant cobblestone epithelial monolayer and EB-like spheroid structures. Immunohisto-fluorescent staining revealed that the EB-like spheroids cells expressed Alkaline phosphatase, SSEA-3, SSEA-4, TRA-1-60, TRA-1-81 (FIG. 5). Expression of the stem cell markers was restricted to the EB-like structures; while more differentiated cells in the epithelial monolayer surrounding the EB-like structures did not react with the antibodies. These data indicate that like ES cells, cultured PSCs form EB-like structures which retain stem cell characteristics.

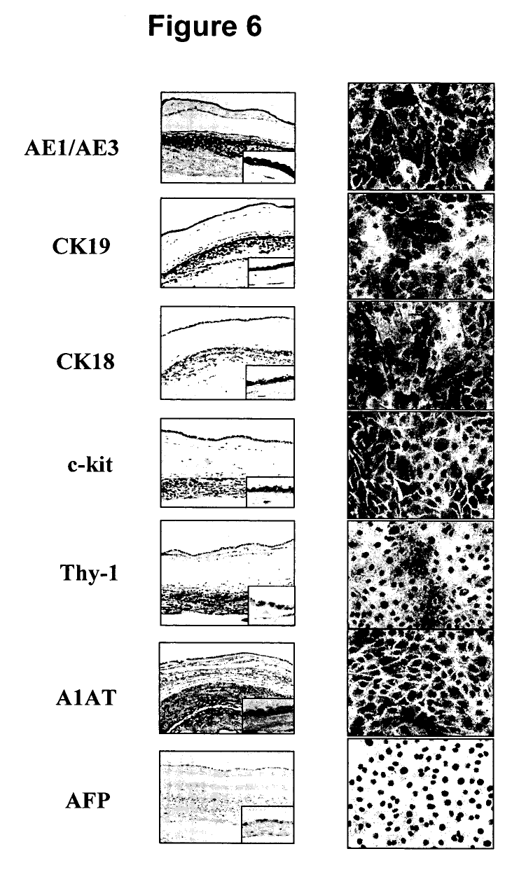

Immunohistochemical analysis on placental tissue and cultured placental stem cells further demonstrated that both the amniotic tissue, the small single row of cells located on the upper side of the placental tissue and the isolated and cultured cells reacted strongly with antibodies to a mixture of cytokeratins (AE1/AE3) and antibodies to cytokeratins 18 and 19, indicating that the placental stem cells are epithelial (FIG. 6). Expression of cytokeratins 8 and 18 are markers of cells of hepatocyte lineage. Cytokeratin 19 expression in liver cells is characteristic of a biliary lineage. Cultured human placental-stem cells also reacted strongly with anti-c-kit antibodies, suggesting that these cells express this growth factor receptor. c-kit, the receptor for the hematopoietic growth factor, stem cell factor (SCF), is expressed by hematopoietic and liver stem cells.

The amniotic tissue is negative for alpha-fetoprotein (AFP) expression, while the cultured cells are very weakly positive for AFP expression. AFP is the fetal form of albumin and is expressed by fetal hepatocytes before they mature. These results contrast with the report of Sakuragawa et al. wherein cultured cells were shown to express AFP (Sakuragawa et al. J Hum Genet 45:171-176 (2000)).

Cultured placental stem cells were also observed to express albumin, a marker of hepatocyte differentiation. Localization of albumin in the population clearly indicates that there are cells which are strongly positive for albumin expression next to cells which are completely negative. Up to 30% of the cultured placental derived cells expressed albumin. It is interesting that some of the strongly albumin positive cells were binucleated, a characteristic of mature hepatocytes. To confirm the expression of albumin at the RNA and protein level. RT-PCR and Western blot analysis were performed using cultured cells. Human albumin RNA was detected using RT-PCR from RNA isolated from cultured cells and human albumin protein was also detected using Western blot on cell extracts from cultured cells.

The cultured placental stem cells reacted with the antibody to alpha-1-antitrypsin (A1AT), while the amniotic tissue was very weak or negative for A1AT expression. A1AT is a protein expressed and secreted by mature hepatocytes and is a marker of hepatocyte differentiation. A1AT was also detected in cell extracts from cultured cells using Western blot analysis. Cell extracts prepared from amniotic tissue, however, did not react with antibodies to albumin or A1AT, suggesting that amniotic tissue does not express albumin or A1AT in vivo. These results suggested that cells cultured under the conditions specified above proliferate and differentiate along the hepatic lineage. These data indicated that cultured placental stem cells are not “locked” into a differentiated state in vivo, but rather, that gene expression in these cells are of a plastic nature.

TABLE 1
Summary of the expression of various markers in cultured
placental stem cells and human placental tissue

| | | |
|---|---|---|
| Epithelial | AE1/AE3 | Cytokeratins, expressed in liver |
| Markers | CK 18 | Cytokeratin, expressed in liver |
| | CK 19 | Cytokeratin expressed in liver |
| | | biliary and liver stem cells |
| Hepatocyte | A1AT | Alpha-1 antitrypsin |
| Markers | Alb | Albumin |
| | AFP | Alpha fetoprotein, liver |
| | | progenitor cells |
| | CYP450 genes | Drug metabolizing enzymes |
| | CYP1A1, 1A2, | expressed in differentiated liver |
| | 2B6, 2C8, | |
| | 2C9, 2D6, 3A4 | |
| Stem | c-kit, Thy-1, | expressed on or by stem cells |
| Cell | SSEA-3, SSEA- | |
| Markers | 4, TRA1-60, | |
| | TRA1-81, Oct-4, | |
| | SOX2, Lefty A, | |
| | FGF-4, Rex-1, TDGF-1 | |

There are master switches which control the pathways through which cells differentiate. Among the most important steps which regulate gene expression along certain lineages are the expression of tissue enriched transcription factors. In liver development, the expression of the Hepatocyte Nuclear Factors (HNF) are such genes. Along the hepatocytic lineage the expression of liver specific genes such as albumin are controlled by HNFs binding to the albumin promoter. In hepatocellular carcinoma cell lines which lack the expression of HNF1 and HNF3, there is no evidence of hepatocyte differentiation. However, transfection of HNF4 activates HNF1 expression and liver specific gene expression (Spath and Weiss. Mol. Cell Biol 17: 1913-1922 (1997)).

Expression of Hepatocyte Nuclear Factor 1 and 4 (HNF 1 and 4 respectively) in cultured placental stem cells was analyzed using immunohistochemical analysis. HNF4 was localized to the nucleus in both human hepatocytes and in the cultured cells. Approximately 25% of the cells exhibited detectable HNF4. Similar results were obtained with HNF1. This relative proportion of cells correlated with the proportion of albumin positive cells described above. These results also provided strong support for the plasticity of cultured placental stem cells, i.e. that these cells can express the transcription factors and the genes required for full hepatic function.

HNF4 expression is not restricted to the liver. HNF4 expression is critical to development and differentiation in the gut, kidney, intestines and pancreatic islets. HNF4 is an important regulator of differentiation in pancreatic beta cells and is critical to the normal development of the pancreatic beta cells. The observations that the cultured placental stem cells express HNF4 indicates that the cultured cells may also have the ability to differentiate into insulin producing beta cells.

### Example 3

### Comparison of Two Different Isolation and Culture Conditions of Placental Stem Cells

Placental stem cells of the invention were cultured in the media as presented in Table 2. The cell isolation and culture conditions which differ from those described by Sakuragawa, et al. (Sakuragawa et al. J Hum Genet 45:171-176 (2000)) and also listed in Table 2. The techniques vary in the concentrations of trypsin, digestion times, culture media and media supplements in the basal media (Table 2).

For the isolation of placental stem cells described in this example, the cells were isolated from the same placenta using the two different techniques. Cells were cultured approximately 10 days in their respective culture media.

TABLE 2
Comparison of Culture medium conditions of
Sakuragawa et al (J Hum Genet. 45: 171-176 (2000))
and Media as used in Example 2.

| | Media of Sakuragawa et | Culture medium |
|---|---|---|
| | al. (J Hum Genet | conditions of present |
| | 45: 171-176, 2000) | invention |
| Trypsin Conc. | 0.1% | 0.05% |
| Digestion time | 15 min | 30 min × 2 |
| Culture media | RPMI | DMEM (high glucose) |
| Supplement | 10% FBS | 10% FBS |
| | | EGF |
| | | Sodium pyruvate |
| | | 1% non essential amino acid |

The differences in cell isolation and culture may lead to the isolation of cell types different from those isolated and/or propagated using the Sakuragawa technique (J Hum Genet. 45:171-176 (2000)).

To determine the expression of specific genes in culture, real time quantitative real time PCR analysis was performed. RNA was isolated from the placental stem cells cultured in two different cell culture media (Table 2) and examined by quantitative PCR for gene expression. Real time PCR is a process where quantitative analysis of gene expression can be accomplished by doing a normal PCR reaction and measuring the product produced in real time using a fluorescent dye. The dye is in excess in the reaction so that when it interacts with DNA the fluoresces is in proportion to the amount of DNA. It is by this mechanism that one can get a quantitative measurement of the amount of RNA or DNA in the original solution. For RNA quantitation one begins with a reverse transcriptase step to convert RNA into DNA which can then be amplified through regular PCR. These assays are conducted on a real-time PCR machine supplied by Applied Biosystems and a complete protocol for quantitative PCR is supplied as product numbers 4310251 and 4304449. In each case the relative level of expression of the indicated gene is compared to the expression of β-actin, the internal control.

To address previously reported isolation and culture conditions, the expression of a large number of genes under the conditions of the present invention and those of Sakuragawa (J Hum Genet. 45:171-176 (2000)) were compared (Table 2). Isolated RNA from the cells were analyzed on a gene array. These arrays contain DNA sequences specific for thousands of genes, such that an analysis of gene expression of several thousand genes can be conducted at one time. Two arrays were run. One with the RNA from the cells isolated and cultured under the methods of Sakuragawa (J Hum Genet. 45:171-176 (2000)) and another with the cells isolated from the same placenta using the conditions of present invention (Table 2). Cells were cultured under each condition for two weeks. Cells were scraped and spun down at 1000 rpm for 5 min. The pelleted cells were snap-frozen with liquid nitrogen and stored in −80° C. until analysis. Total RNA was extracted and mRNA was purified to hybridize to DNA microarrays (Affymetrix U133A). Scanned arrays were analyzed with Affymetrix MAS 4.0 software to identify genes which were expressed at different levels between the two conditions.

Results

Expression of Liver-Specific Markers

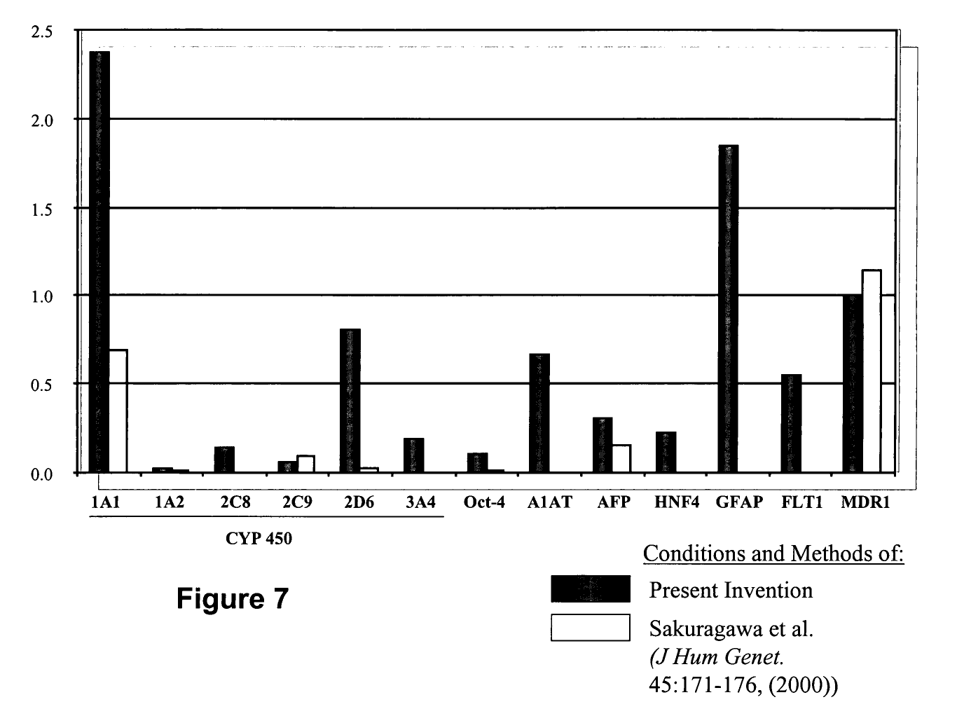

The expression of several liver-specific genes in placental stem cells cultured using the conditions of Sakuragawa et al (J Hum Genet. 45:171-176 (2000)) or the methods of the present invention were examined using real time PCR. The cultured cells were examined for expression of the following liver specific genes, cytochromes such as CYP1A1, CYP1A2, CYP2C8, CYP2C9, CYP2D6, CYP3A4; Oct 4, A1AT, AFP, HNF4, GFAP, FLT1, and MDR1 (see FIG. 7). The CYP genes code for drug metabolizing enzymes expressed in the liver. Of the 13 different genes examined, only MDR1, or the multidrug resistance gene and CYP2C9 were expressed at similar levels between the culture conditions of Sakuragawa (J Hum Genet. 45:171-176 (2000)) and the conditions of the present invention. The cultured cells exhibited significant differences in gene expression, in particular, for CYP1A1, CYP 2C8, CYP2D6, and CYP3A4. This disparity suggest that the cells cultured using the method of the present invention demonstrate a far superior ability to differentiate into hepatocytes in comparison to cells isolated using the method of Sakuragawa et al (J Hum Genet. 45:171-176 (2000)).

Other genes, such as Oct-4, alpha-1 antitrypsin (A1AT), GFAP and FLT-1, are also expressed only under the culture conditions of the present invention. A1AT and HNF-4 are markers of differentiated hepatocytes. The liver produces and secretes A1AT and HNF4 is a transcription factor required for the maintenance of differentiated liver function. GFAP is glial fibrillary acid protein, a marker for neuronal glial cells and FLT-1 is a surface receptor expressed on vascular endothelial cells. Both GFAP and FLT-1 are detectable in the placental stem cells isolated and cultured under the conditions of the present invention. Their expression in placental stem cells suggest that these cells can differentiate along neuronal and endothelial lineages, as well as towards hepatocyte cell lineages. It is not clear whether these markers, commonly found on different tissue types, are expressed on the same cells or on different cells within our cultures. The presence of markers of differentiated neuroglial cells, differentiated hepatocytes and vascular endothelial cells in the same cultures may indicate that the isolation conditions of the present invention provide a means for the isolation of cells having different differentiation potentials. Alternatively, the media and growth conditions of the present invention may provide a wider range of differentiation potential from the same cell type.

One argument for the isolation of different cell types is the observation of the presence of Oct-4 positive cells only using the isolation and culture conditions of the present invention. Expression of Oct-4 is thought to be restricted to totipotent stem cells such as the ES cells. The presence of Oct-4 in the cell cultures of the present invention, but not that of Sakuragawa et al. (J Hum Genet. 45:171-176 (2000)) indicates the isolation of a different cell type by the isolation conditions of the present invention or the rapid loss of this cell type from cultures obtained using the Sakuragawa technique.

An analysis of gene array experiments showed that a total of 2929 genes were expressed at significantly different levels between the culture conditions of the present invention and those of Sakuragawa (J Hum Genet. 45:171-176 (2000)). In this analysis, 885 genes showed an elevated expression under the culture conditions of the present invention while 2044 genes were expressed at lower levels as compared to those under Sakuragawa's (J Hum Genet. 45:171-176 (2000)) conditions. Since the human genome only contains about 30,000 genes and a tissue such as the liver may only express 5,000 total genes, a differential expression of 2923 genes is a large proportion of the total expressed genes. A table of the top fifty genes which were significantly up-regulated under the culture conditions of the present invention and the Sakuragawa (J Hum Genet. 45:171-176 (2000)) conditions are summarized in Tables 3 and 4 respectively. Table 5 lists genes which are also significantly up-regulated in placental stem cells cultured under the conditions of the present invention that are beyond the top fifty up-regulated genes listed in Tables 3 and 4. These selected genes contain many important genes for neural, liver, pancreatic and intestinal cells.

Many of the up-regulated genes were hepatocyte specific or liver related genes (Table 3 and 4). The genes were ranked by the signal intensity in Log scale, so that a gene shown as 1.0 is expressed at 10 times the level compared to the other condition, and a number of 3 would indicate the gene was expressed at 1000 times (or ten to the third power) different levels between the 2 conditions. It is clear that many of the liver related genes are expressed at levels that are 10,000 to 100-million times higher (8.0 in Table 3) under the culture medium conditions of the present invention as compared to those of Sakuragawa (J Hum Genet. 45:171-176 (2000)). Seventeen genes (34% of top 50 genes) that were up-regulated under the present culture medium conditions were liver related, on the other hand only one gene that could be considered as liver related was up-regulated in Sakuragawa's conditions.

TABLE 3
Up-regulated genes in PSCs cultured in Medium of the present invention

| Relate to | Log ratio | Gene description |
|---|---|---|
| O | 8.7 | maltase-glucoamylase (alpha-glucosidase) (MGAM) |
| L | 8.0 | NADP-dependent malic enzyme |
| N | 7.3 | carboxylesterase 3 (brain) (CES3) |
| L | 7.2 | transthyretin precursor |
| L | 6.4 | aldolase B, fructose-bisphosphate (ALDOB) |
| L | 6.3 | complement component 3 (C3) |
| L | 6.3 | cytochrome P450-2E1 (CYP2E1) |
| A | 6.2 | FK506-binding protein 5 (FKBP5) |
| L | 6.0 | glycine N-methyltransferase (GNMT) |
| L | 5.5 | 4-hydroxyphenylpyruvate dioxygenase (HPD) |
| L | 5.5 | insulin-like growth factor I (IGF-I) |
| L | 5.4 | epoxide hydrolase 1, microsomal (xenobiotic) (EPHX1) |
| N | 5.4 | phenylethanolamine N-methyltransferase (PNMT) |
| O | 5.2 | NOD2 protein (NOD2) |
| P | 5.2 | protease, serine, 22 (P11) |
| A | 5.1 | Na+H+ exchanger isoform 2 (NHE2) |
| O | 4.9 | S100 calcium-binding protein A4 (S100A4), transcript variant 1 |
| A | 4.8 | haptoglobin-related protein (HPR) |
| N | 4.8 | huntingtin-associated protein interacting protein (duo) (HAPIP) |
| O | 4.8 | sodium channel, nonvoltage-gated 1 alpha (SCNN1A) |
| L | 4.7 | fatty acid binding protein 1, liver (FABP1) |
| L | 4.6 | cytochrome P450, subfamily XXVIA, polypeptide 1 (CYP26A1) |
| O | 4.6 | epithelial membrane protein 1 (EMP1) |
| O | 4.4 | Pur-gamma (PURG) |
| N | 4.3 | cadherin 18, type 2 (CDH18) |
| A | 4.3 | solute carrier family 2 (facilitated glucose transporter), member 5 (SLC2A5) |
| L | 4.2 | bile salt export pump (BSEP) |
| A | 4.2 | desmoglein 1 (DSG1) |
| A | 4.2 | potassium inwardly-rectifying channel, subfamily J, member 16 (KCNJ16) |
| A | 4.2 | solute carrier family 14 (urea transporter), member 1 (Kidd blood group) (SLC14A1) |
| N | 4.1 | amphiregulin (schwannoma-derived growth factor) (AREG) |
| O | 4.0 | fatty acid binding protein 3, muscle and heart (FABP3) |
| A | 3.9 | nidogen 2 (NID2) |
| O | 3.9 | retinoid X receptor, gamma (RXRG) |
| L | 3.8 | ceruloplasmin (ferroxidase) (CP) |
| O | 3.8 | killer cell inhibitory receptor homolog cl-9 |
| A | 3.8 | regulator of G-protein signalling 2, 24 kD (RGS2) |
| O | 3.7 | kidney-enriched Kruppel-like factor (KKLF) |
| L | 3.7 | short-chain alcohol dehydrogenase family member (HEP27) |
| L | 3.6 | aminolevulinate, delta-, dehydratase (ALAD) |
| O | 3.6 | epididymis-specific, whey-acidic protein type (HE4) |
| O | 3.6 | peroxisome proliferative activated receptor, gamma (PPARG) |
| O | 3.6 | prolactin receptor (PRLR) |
| O | 3.6 | prostaglandin F receptor (FP) (PTGFR) |
| L | 3.6 | SMP-30 (senescence marker protein-30) |
| A | 3.6 | TATA box binding protein (TBP)-associated factor, RNA polymerase II, Q (TAF2Q) |
| L | 3.5 | fructose-1,6-bisphosphatase |
| P | 3.5 | tissue factor pathway inhibitor beta (TFPIbeta) |
| O | 3.4 | nuclear receptor subfamily 3, group C, member 2 (NR3C2) |
| O | 3.4 | pre-B-cell leukemia transcription factor 1 (PBX1) |

A: All tissues,
L: Liver,
N: Neural,
P: Placenta,
O: others

TABLE 4
Up-regulated genes in PSCs cultured in Medium of Sakuragawa
(J Hum Genet. 45: 171-176 (2000))

| Relate to | Log ratio | Gene description |
|---|---|---|
| A | 10.7 | connective tissue growth factor |
| O | 8.4 | serine proteinase inhibitor, clade E, member 1 (SERPINE1) |
| O | 7.8 | cytolysis inhibitor (CLI) |
| O | 7 | CYR61 |
| O | 6.9 | parathyroid-like protein |
| O | 6.8 | insulin-like growth factor binding protein 7 (IGFBP7) |
| O | 6.7 | L-type amino acid transporter 1 |
| L | 6.4 | heptacellular carcinoma novel gene-3 protein |
| O | 6.2 | myosin regulatory light chain 2, smooth muscle isoform (MYRL2) |
| O | 6.1 | twisted gastrulation (TSG) |
| N | 5.9 | dihydropyrimidinase-like 3 (DPYSL3) |
| N | 5.7 | carboxypeptidase E (CPE) |
| O | 5.6 | hexabrachion (tenascin C, cytotactin) (HXB) |
| O | 5.4 | keratin 17 (KRT17) |
| A | 5.3 | kinesin-like 5 (mitotic kinesin-like protein 1) (KNSL5) |
| A | 5.3 | fibroblast growth factor receptor 2(FGFR2) |
| N | 5.2 | GABA-B receptor |
| A | 5.2 | leucine-zipper protein FKSG13 (FKSG13) |
| O | 5.1 | tropomyosin 2 (beta) (TPM2) |
| A | 5 | fibulin 1 (FBLN1), transcript variant C |
| O | 5 | guanylate binding protein 1, interferon-inducible, 67 kD (GBP1) |
| A | 4.9 | solute carrier family 2, member 3 (SLC2A3) |
| A | 4.9 | G protein-coupled receptor, family C, group 5, member B (GPRC5B) |
| O | 4.8 | ovarian beta-A inhibin |
| O | 4.8 | oxytocin receptor (OXTR) |
| O | 4.8 | transgelin (TAGLN) |
| O | 4.8 | transmembrane 4 superfamily member (tetraspan NET-2) (NET-2) |
| A | 4.6 | transforming growth factor-beta-2 |
| N | 4.6 | bullous pemphigoid antigen 1 (230240 kD) (BPAG1) |
| O | 4.6 | CD24 |
| O | 4.5 | ectodermal-neural cortex (with BTB-like domain) (ENC1) |
| N | 4.5 | calpain 6 (CAPN6) |
| O | 4.4 | tumor-associated calcium signal transducer 1 (TACSTD1) |
| O | 4.4 | lysyl oxidase-like 1 (LOXL1) |
| O | 4.3 | tropomyosin 4 |
| A | 4.3 | eIF4E-transporter (4E-T) |
| A | 4.2 | keratin 14 |
| A | 4.2 | 1,2-cyclic-inositol-phosphate phosphodiesterase (ANX3) |
| O | 4.2 | histamine N-methyltransferase (HNMT) |
| O | 4.2 | putative transmembrane protein (NMA) |
| A | 4.2 | latent transforming growth factor beta binding protein 3 (LTBP3) |
| N | 4.1 | Kallmann syndrome 1 sequence (KAL1) |
| O | 4.1 | serine proteinase inhibitor, clade H, member 1 (SERPINH1) |
| O | 4.1 | wingless-type MMTV integration site family, member 11 (WNT14) |
| O | 4.1 | MAD homolog 7 (MADH7) |
| A | 4 | checkpoint suppressor 1 (CHES1) |
| A | 4 | putative endothelin receptor type B-like protein |
| P | 3.9 | cadherin 3, type 1, P-cadherin (placental) (CDH3) |
| O | 3.9 | CD151 antigen (CD151) |
| O | 3.9 | putative integral membrane transporter (LC27) |

A: All tissues,
L: Liver,
N: Neural,
P: Placenta,
O: others

TABLE 5
Up-regulated genes in PSCs cultured in
Medium of the present invention (Selected)

| Log Ratio | Gene description |
|---|---|
| 3 | ATPase, Na+K+ transporting, beta 1 polypeptide |
| 2.9 | amylase, alpha 1A; salivary (AMY1A) |
| 2.9 | c-mer proto-oncogene tyrosine kinase (MERTK) |
| 2.8 | activin A receptor, type IIB (ACVR2B) |
| 2.8 | albumin/FL |
| 2.7 | neuritin (LOC51299) |
| 2.7 | UDP glycosyltransferase 1 family, polypeptide A3 (UGT1A3) |
| 2.5 | fibrinogen, gamma polypeptide (FGG), transcript variant gamma-A |
| 2.4 | colony stimulating factor 1 receptor (CSF1R) |
| 2.4 | cytochrome P450-2A6 (CYP2A6) |
| 2.4 | transforming growth factor, beta receptor III (betaglycan, 300 kD ) (TGFBR3) |
| 2.3 | ATP-binding cassette, sub-family C (CFTRMRP), member 2 (ABCC2), |
| 2.3 | dipeptidylpeptidase IV (CD26, adenosine deaminase complexing protein 2) (DPP4) |
| 2.3 | estrogen receptor |
| 2.2 | cytochrome P450IIE1 (ethanol-inducible) |
| 2.1 | cytochrome P450, (CYP2D6) |
| 2.1 | ADP-ribosylation factor-like 4 (ARL4) |
| 2.1 | UDP-N-acetyl-alpha-D-galactosamine: (GaINAc-T7) (GALNT7) |
| 2 | cytochrome P450IIA3 (CYP2A3) |
| 1.9 | Jak2 kinase (JAK2) |
| 1.9 | acetyl-Coenzyme A acetyltransferase 2 (acetoacetyl Coenzyme A thiolase) |
| 1.8 | fumarylacetoacetate (FAH) |
| 1.8 | cyclin-E binding protein 1 (LOC51191) |
| 1.7 | insulin-like growth factor 2 (somatomedin A) (IGF2) |
| 1.7 | gamma-aminobutyric acid (GABA) receptor, rho 1 (GABRR1) |
| 1.6 | FGF receptor 4b |
| 1.5 | cytochrome P450-3A4 (CYP3A4) |
| 1.5 | Similar to solute carrier family 1 (glutamate transporter), member 7 |
| 1.5 | preproinsulin-like growth factor II (IGF-II) |
| 1.5 | argininosuccinate lyase (ASL) |
| 1.5 | cytochrome P450, (CYP2A7) |
| 1.5 | fucose-1-phosphate guanylyltransferase (FPGT) |
| 1.5 | cytochrome P450, (CYP7B1) |
| 1.5 | inhibin, beta C (INHBC) |
| 1.5 | mitogen-activated protein kinase kinase kinase 12 (MAP3K12) |
| 1.5 | FLT4 ligand |
| 1.4 | frizzled 1 |
| 1.4 | dopamine receptor D2 (DRD2) |
| 1.4 | ATP-binding cassette, sub-family D (ALD), member 3 (ABCD3) |
| 1.4 | STAT induced STAT inhibitor-2 (STATI2) |
| 1.4 | mucosal vascular addressin cell adhesion molecule 1 (MADCAM1) |
| 1.4 | interleukin 1-beta converting enzyme isoform gamma (IL1BCE) |
| 1.3 | signal transducer and activator of transcription 6, interleukin-4 induced |
| 1.3 | interleukin-1 beta convertase (IL1BCE) |
| 1.3 | estrogen receptor 1 (ESR1) |
| 1.3 | gastrin (GAS) |
| 1.3 | carboxypeptidase A2 (pancreatic) (CPA2) |
| 1.2 | vascular endothelial growth factor (VEGF) |
| 1.2 | glutathione S-transferase A4 (GSTA4) |
| 1.2 | organic cationic transporter-like 4 (ORCTL4) |
| 1.2 | urokinase-type plasminogen activator receptor |
| 1 | frizzled (Drosophila) homolog 1 (FZD1) |
| 0.9 | adipose differentiation-related protein |
| 0.9 | keratin 19 (KRT19) |
| 0.9 | neuroendocrine secretory protein 55 (NESP55) |
| 0.8 | thiopurine methyltransferase (TPMT) |

### Example 4

### Optimal Media Conditions for Enhanced Expression of Liver Specific Genes in Placental Stem Cells

Placental stem cells were isolated as described in Example 2 and cultured using basal culture conditions found in Table 2 for cell plating and expansion of the cells for 10-14 days. The cells were cultured for either 7-10 days or until the cultures grew to confluence. The cells were trypsinized and reseeded in 6 well plates. The cells were subjected to different culture conditions, having varying growth factors supplementing the DMEM or MEM based medium. The following media combinations were used: DMEM and 10% FBS was supplemented with either no additional growth factors or; Epidermal Growth Factor (EGF) alone; or EGF and dexamethasone (DEX) or; EGF+DEX+hepatocyte growth factor (HGF)+Insulin-Transferrin-Selenium (ITS) or; EGF+DEX+Fibroblast Growth Factor 2 (FGF-2)+ITS or; EGF+DEX+FGF-4+ITS or; EGF+DEX+FGF-7+ITS or; EGF+HGF. Placental stem cells were cultured for an additional 14 days. At the end of 14 days, the cells were evaluated for the expression of human albumin, CYP3A4, A1AT, or C/EBP-alpha.

RT-PCR was also run on RNA isolated from the cells and performed as described in previously.

Results

A panel of media supplemented with various growth factors and/or combinations of growth factors were used to culture the placental stem cells to identify optimal culture media for enhanced expression of liver specific genes. The cultured cells were analyzed for expression of human albumin, CYP3A4, A1AT, and C/EBP-alpha. The results indicate that under certain culture conditions the expression of albumin, CYP3A4, A1AT and C/EBP alpha increase considerably over the initial values reported. In particular, the inclusion of EGF and dexamethasone (Dex) was shown to enhance liver specific gene expression. At least at these time points in culture, the additional supplementation of the media with hepatocyte growth factor (HGF), or the fibroblast growth factors (FGF) 2,4, or 7 did not enhance liver specific gene expression. The data obtained indicated that modification of the culture conditions from the basal growth media listed in Table 2 to the differentiation media conditions indicated above can enhance the expression of liver specific genes. These results suggest that liver specific gene expression is enhanced by the use of the differentiation media described herein. While HGF and the fibroblast growth factors did not appear to enhance differentiation along the hepatocyte pathway, these growth factors may promote differentiation of cultured cells to other cell types such as neuronal, pancreatic or muscle cell differentiation.

### Example 5

### Differentiation of Placental Stem Cells into Hepatocytes

Freshly isolated PSCs were allowed to proliferate for one week, and sub-cultured with 4×10³ cells/cm² cell density on a Type-I collagen-coated plate. Dexamethasone and insulin (0.1 μM) were added in the culture medium to enhance hepatic differentiation. Phenobarbital (1 mM) was added for the final 3 days and RNA was isolated and real time quantitative PCR was performed as described in Example 3. Immunohistochemical analysis for human HNF-4 alpha and albumin was prepared with rabbit anti-human HNF-4 alpha and anti-human albumin respectively.

Results

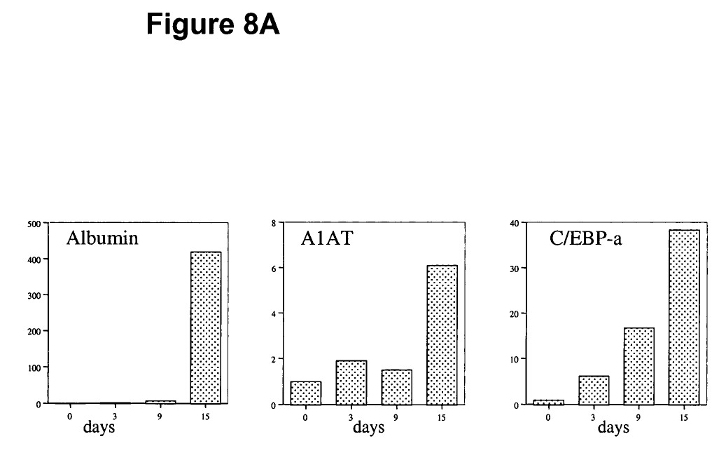

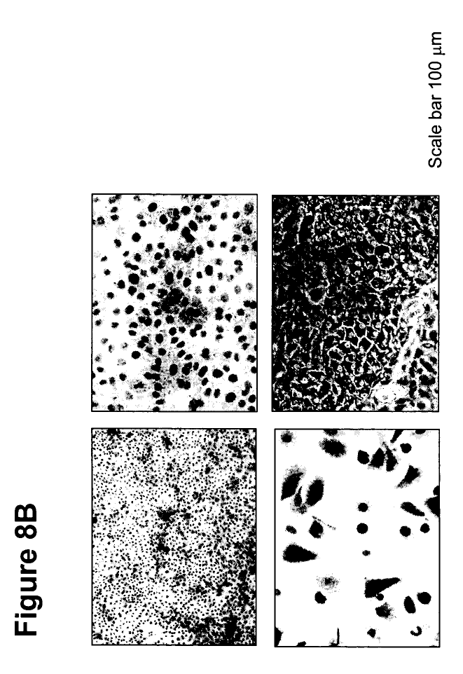

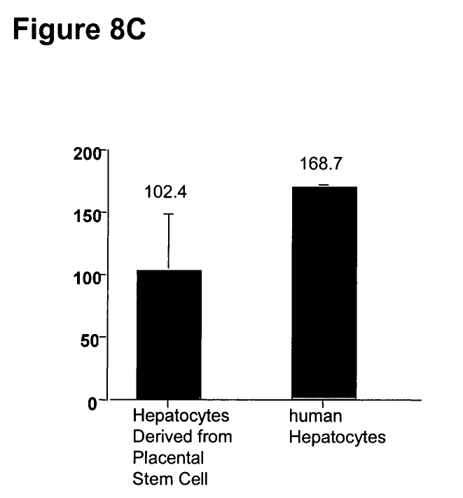

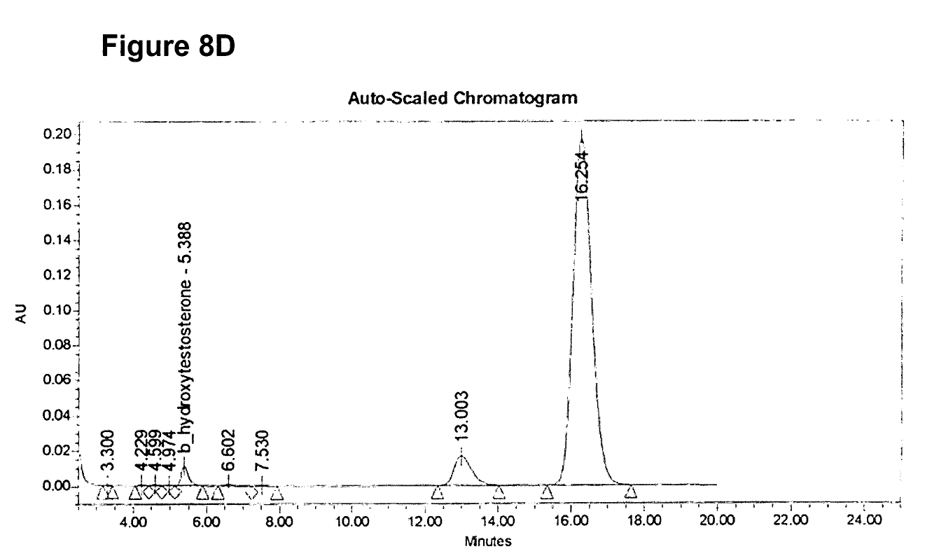

Characteristic hepatocyte genes, albumin (Alb) and alpha-1-antitrypsin (A1AT), and the transcription factor, C/EBPa, mRNA expression was examined by real-time quantitative PCR (FIG. 8A). The results indicate that genes characteristic of hepatocytes such as A1AT, C/EBPa, and albumin are increased as much as 500-fold as the cells differentiate. To confirm expression of prototypical liver genes, PSC derived hepatocytes were immunostained with anti-human serum albumin antibody and anti-HNF-4a antibodies (FIG. 8B). At this time in culture, approximately 33% cells are positive for albumin and some are strongly positive for human albumin. Most of these strongly positive cells are bi-nucleated cells which resemble normal human hepatocytes. In separate experiments nuclear localization of HNF-4a, was observed and additional cells showed strong cytoplasmic staining with anti-HNF-4a, suggesting differentiation was taking place in these cells and that nuclear localization of HNF-4a, would follow in time. Longer term culture revealed clusters of small cells with refractile cell junctions with the morphology of human hepatocytes in primary culture (FIG. 8B).

### Example 6

### Metabolic Function of Hepatocytes Derived From Placental Stem Cells

Human hepatocytes were cultured using the conditions of Strom et al. Methods in Enzymology 272:388-401 (1996). Placental stem cells were differentiated into hepatocytes by culturing the stem cells in media supplemented with 10 ng/ml EGF, 0.1 μM Dexamethasone, 10 μg/ml Insulin, 5.5 μg/ml Transferrin, 6.7 ng/ml Selenium, and 2 μg/ml Ethanolamine.

An EROD assay which measures the conversion of ethoxy-resorufin to hydroxyresorufin was used to detect expression of CYP1A1 or CYP1A2 in human hepatocytes and hepatocytes derived from placental stem cells (Kelley et al. J. Biomolecular Screening 5:249-253 (2000). CYP1A activity in these cells was induced by exposing the hepatocytes to beta-naphthoflavone (50 uM).

The expression of CYP3A4 in the liver was measured as the specific conversion of testosterone to the 6-beta-hydroxy metabolite (Kostrubsky et al. Drug Metab. Dispos. 27:887-894 (1999).

Uptake of Indocyanine Green (ICG) is another clinical test that was utilized to assay for hepatocyte function. In patients, ICG is injected into the blood stream and as it passes through the liver the dye is taken up by transport proteins specific to the liver. The transporter proteins involved in the uptake of ICG are called OATP (organic anion transporter protein) and a liver specific organic anion transporter (LST).

Results

Normally analysis of CYP1A1 and CYP3A4 activity in human hepatocytes is accomplished by measuring the ability of the cells to metabolize drugs or specific compounds which are substrates for different CYP450 genes. The levels of these enzymatic processes in hepatocytes derived from placental stem cells (PSCs) were evaluated using three methods: the EROD assay, by determining the presence of 6-beta-hydroxy metabolite, and by observing the uptake of indocyanine green.

Results from the ethoxyresorufin assay showed that hepatocytes derived from PSCs metabolize ethoxyresorufin. For comparison, the EROD assay was also performed on authentic human hepatocytes isolated from a donor liver not used for whole organ transplantation. As with human hepatocytes, the hepatocytes derived from PCS do not express much enzymatic activity under basal conditions. With both the hepatocytes and the hepatocytes derived from PSCs, EROD activity is induced by prior exposure of the cells to beta-naphthoflavone (BNF). Beta-Naphthoflavone was chosen for this study based on its ability to stimulate CYP1A1/2 expression in the liver and in cultured hepatocytes. The data indicate that the expression of CYP1A1/2 in the hepatocytes derived from PSCs is equal to approximately 60% of the activity seen in authentic human hepatocytes.

Similar results were observed using hepatocytes derived from placental stem cells that were cultured on Type-I collagen-coated plate supplemented with Dexamethasone (0.1 μM) and insulin (0.1 μM) (FIG. 8C).

In experiments to determine the metabolism of testosterone, high pressure liquid chromatographic (HPLC) separation of testosterone metabolites generated in placental stem cell-derived hepatocytes demonstrates clearly the production of 6-beta-hydroxytestosterone by hepatocytes derived from PSCs (FIG. 8D). Hepatocytes derived from PSCs not only express RNA for the specific P450 genes, but that the cells actually translate the protein and make active drug metabolizing enzymes. The presence of such metabolic functions confirm the usefulness of such cells for drug metabolism or toxicology studies, artificial liver devices or for clinical hepatocyte transplants.

The uptake of ICG by hepatocytes derived from PSCs was also examined to determine the hepatocytes derived from PSCs exhibited true hepatocyte function. 13.9% of the hepatocytes derived from PSCs show uptake of ICG in comparison to 46.4% in human hepatocytes. These data indicate the presence of liver specific drug and chemical transporters on the hepatocytes derived from PSCs and further establish the utility of the hepatocytes derived from PSCs for drug metabolism and toxicology studies as well as artificial liver devices and hepatocyte transplants.

### Example 7

### Transplantation of Cultured Placental Stem Cells into Mouse Liver and Differentiation of Said Cells to Human Hepatocytes

Two million placental stem cells were transplanted into the liver via the spleen. Pictures were taken one month following transplantation. Because of bleeding difficulties following direct transplantation of cells into liver or portal vein, it has been established that approximately 50% of the cells transplanted into the spleen will translocate to the liver within 5 minutes (Ponder et al. Genetics 88:1217 (1991)). Once in the liver, transplanted hepatocytes incorporate into hepatic plates and survive long-term.

In one experiment, placental stem cells were first infected with an adenovirus vector containing GFP to label the cells prior to transplantation. At the time of transplantation >85% of the placental stem cells were labeled with the green fluorescent protein. Recipient animals were SCID or Rag-2 knock out animals. These mouse strains are immunocompromised and are regularly used for investigations of the transplantation of human tissues or cells because the animals do not readily reject the foreign tissue/cells.

Results

Results from this experiment demonstrate that placental stem cells translocate to the liver from the spleen, integrate into hepatic plates and express the morphology of hepatocytes and genes associated with normal liver. Placental stem cells that were labeled with a viral vector expressing Green Fluorescent Protein (GFP) were observed in sections of the liver of these animals. The fluorescently labeled cells exhibit the morphology of normal hepatocytes which have been incorporated into hepatic plates. Cells which do not incorporate into hepatic plates die and are rapidly removed from the liver my macrophages within 3-7 days, so the results observed here represent only those cells which have become stably incorporated into the mouse liver. The frequency of integration of the placental stem cells into the liver can be calculated from the number of labeled cells recovered from the liver. The frequency of integration is very high for hepatocytes derived from PSCs as compared to normal hepatocytes. In published reports, integration frequencies of transplanted hepatocytes range from 0.1% to 10%. The integration of hepatocytes derived from PSCs is approximately 51%. These data indicate that the placental stem cells will be useful for hepatocyte transplantation studies.

Transplants of actual human hepatocytes into immunocompromised mice provide virtually identical results to that shown here following the transplantation of the placental stem cells (Dandri et al. Hepatol. 33:981-988 (2001) and Mercer et al. Nature Med. 7:927-933 (2001)). These data indicate that the transplanted placental stem cells mature to human hepatocytes in the liver of the recipient.

At 1 month following transplantation of placental stem cells into the liver of immunodeficient mice, these animals were sacrificed and sections were made of the liver of transplanted animals. Liver sections of transplanted cells were examined for the presence of human alpha-1 antitrypsin or human albumin with antibodies. Results demonstrate that cells with the morphology of hepatocytes in the liver sections react with antibodies to human A1AT or human albumin. These data confirm that human placental stem cells transplanted into the liver of immunodeficient mice integrate into the hepatic plates, have the morphology of normal human hepatocytes and express genes usually expressed in normal human liver.

### Example 8

### Differentiation of Placental Stem Cells into Neural and Vascular Endothelial Cells

Placental stem cells were cultured in the presence of FGF-4 (10 ng/ml) for approximately 14 days. Immunohistochemical analysis of the expression of the different genes was conducted with antibodies specific to the human proteins.

Alternatively, freshly isolated PSCs were also cultured in media supplemented with all-trans retinoic acid (a differentiation program used for neural stem cells (Kukekov et al. Exp. Neurol. 156, 333 (1999) and Vescovi et al. Exp. Neurol. 156, 71 (1999))) for 7 days with essentially similar results.

To differentiate the placental stem cells into vascular endothelial cells, the cells were cultured for approximately 10 days in both growth mode and plated on dishes coated with Matrigel™ (20T on 100% u/u). Placental stem cells were cultured on Matrigel as also disclosed in Grant et al and Kazuya et al. (Grantet al. In Vitro Cell Dev. Biol. 27A: 327-336 (1991); and Kazuya et al. J Cell Physiol. 161: 267-276 (1994)).

Results

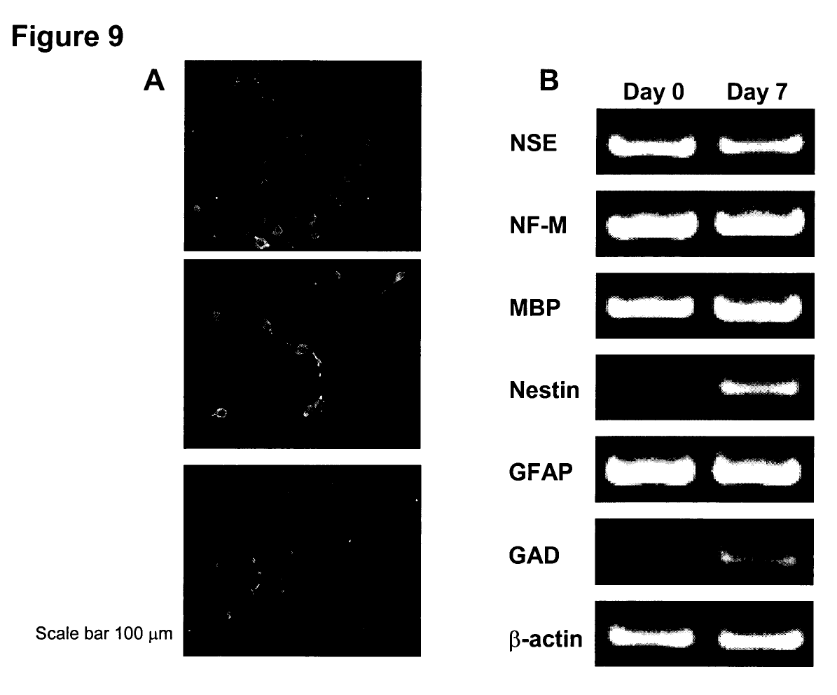

Results from this experiment demonstrate that placental stem cells cultured in the presence of all trans retinoic acid, express glial fibrillary acidic protein (GFAP)— a marker of oligodendrocytes, beta-tubulin III—a marker for astrocytes, and C-type natriuretic peptide (CNP)—a marker for neurons (FIG. 9A). Similar results were obtained in placental stem cells that were cultured in the presence of FGF-4.

Many of the neural specific genes such as NSE, NF-M, MBP, GFAP are expressed even in the freshly isolated cells (day 0), while the expression of nestin and glutamic acid decarboxylase (GAD) the rate-limiting enzyme in GABA biosynthesis increase over 7 days of being cultured in media containing trans-retinoic acid (FIG. 9B). These results indicate that the cultured placental stem cells are multipotent, that is, they can differentiate along the neuronal, oligodendrocyte and astrocytic lineages.

On a culture substrate called Matrigel, placental stem cells aggregate into web-like formations and form tubules with open lumens (FIG. 10). These morphologic changes indicate the first steps in the development of vascular channels. Cells on matrigel and observed web-like formation reminiscent of authentic vascular endothelial cells are shown in FIG. 10. At higher power (400×) the elongated capillary-like structure is clearly observed (FIG. 10). A transmission electron micrograph (4,000×) shows the rudimentary formation of a vascular channel (FIG. 10). These data indicate that cultured placental stem cells can differentiate along an endothelial cell pathway and can be used as stem cells for the formation, reconstruction or repair of the human vascular system.

### Example 9

### Differentiation of Placental Stem Cells into Pancreatic Cells

Placental stem cells were maintained in standard growth media for 7 days and then trypsinized and seeded on cultures previously coated with matrigel (MG), a commercially available form of basement membrane proteins. Cultures were coated with 20% (v/v; matrigel to media) or 100% matrigel with essentially identical results. Cells were seeded on plates previously coated with MG and were cultured an additional 14 days in Matrigel supplemented with Dexamethasone (0.1 micromolar) and the standard concentrations of ITS or in Matrigel supplemented with nicotinamide (10 mM). After 10-14 days the cells were lysed and RNA was isolated, and reverse transcriptase-PCR analysis was conducted with PCR primers specific for Pax 6, PDX-1 Nkx2.2, insulin, glucagon and an internal control, beta-actin.

Results

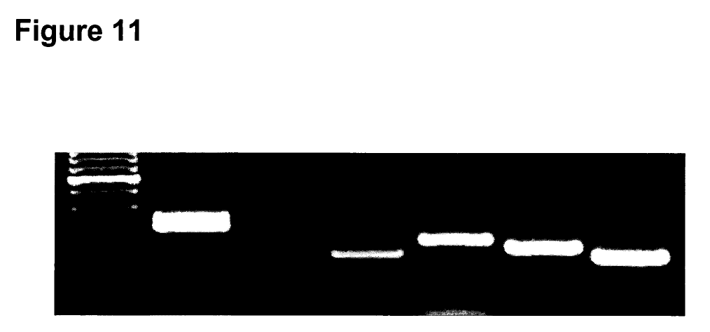

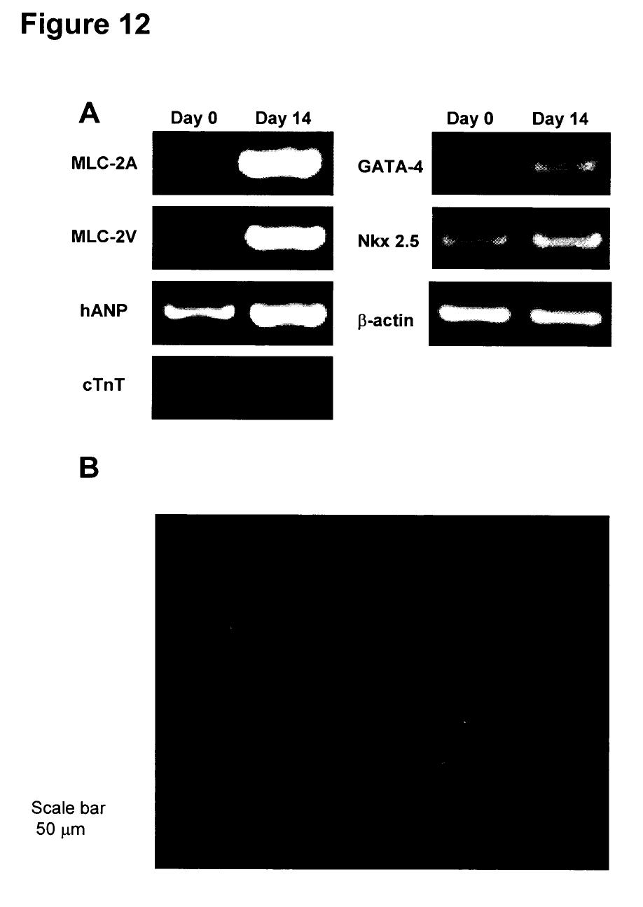

As shown in the RT-PCR analysis (FIG. 11), placental stem cells cultured in Matrigel supplemented with 10 mM nicotinamide for 14 days, express insulin and glucagon as well as the usual lineage transcription factors, PDX-1 (faint), Pax6 and Nkx2.2. Prior to transfer of the cells to matrigel, PDX-1 expression is much higher than shown in FIG. 11, and Pax 6, Nkx2.2, insulin and glucagon expression was not observed (data not shown) suggesting that the additional culture treatments enhanced the pancreatic differentiation of PSC. Similar results were observed in placental stem cells cultured in Matrigel supplemented with Dexamethasone (0.1 micromolar) and standard concentrations of ITS.

### Example 10

### Differentiation of Placental Stem Cells into Cardiomyocytes

Placental stem cells (PSCs) were cultured in the presence of L-Ascorbic acid 2-phosphate (1 mM) for 14 days. Total RNA was extracted on day 0 and day 14 and used for RT-PCR analysis. Cardiomyocyte specific genes primers were designed specifically for cardiac transcription factor GATA-4, atrial myosin light chain type 2 (MLC-2A), ventricular myosin light chain type 2 (MLC-2V), human atrial natriuretic peptide (hANP), and cardiac troponin T (cTnT). The PCR reaction conditions were 45 cycles/57° C. annealing temperature. The PCR products were size fractioned by 2.5% agarose gel electrophoresis.

Results

Results of this experiment showed that PSCs at day 0 and differentiated PSCs at day 14 both express the cardiac transcription factor GATA-4. GATA-4 is expressed in precardiac mesoderm and persists in the heart during development. MLC-2A and MLC-2V genes are widely used to determine cardiomyocytes derived from embryonic stem cells (Kehat et al. J Clin Invest 108, 407-14, 2001). In this experiment these genes were significantly up-regulated in differentiated PSCs which means at least some of the cells were differentiated specifically into cardiomyocytes.

ANP is a hormone that is actively expressed in both atrial and ventricular cardiomyocytes in developing heart, but is significantly down-regulated in adult ventricular cells (Zeller et al. Genes Dev. 1, 693-8 (1987)). Cardiac specific troponin T is a subunit of the troponin complex that provides a calcium sensitive molecular switch for the regulation of striated muscle contraction (Xu et al. Circ Res. 91, 501-8 (2002)).

These data indicate that the PSCs differentiate towards cardiomyocytes (mesoderm). This is proof of concept that PSCs can become cardiac muscle. It is considered likely that even relatively immature cells committed to cardiac differentiation would be useful in a transplantation setting to restore impaired cardiac function (Nir et al. Cardiovasc Res. 58, 313-23 (2003)). Immunohistochemical analysis further demonstrates that PSC-derived cardiac muscle cells express alpha actinin—a marker of cardiac muscle development which is frequently used to demonstrate cardiac differentiation of ES cells.

The practice of the present invention will employ, unless otherwise indicated, conventional techniques of cell biology, cell culture, molecular biology, recombinant DNA, and immunology, which are within the skill of the art. Such techniques are described in the literature. See, for example, Molecular Cloning: A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Antibodies: A Laboratory Manual, and Animal Cell Culture (R. I. Freshney, ed. (1987); Culture of Animal Cells, A Manual of Basic Technique, 2d Ed., (R. I. Freshney, A. R. Liss, Inc., New York, 1987); Culture of Epithelial Cells (R. I. Freshney ed, Wiley-Liss, 1992), Embryogenesis in vitro: Study of Differentiation of Embryonic Stem Cells. Biol Neonate (Vol 67:77-83, 1995); Cell Therapy: Stem Cell Transplantation, Gene Therapy, and Cellular Immunotherapy (G. Morstyn & W. Sheridan eds, Cambridge University Press, 1996); and Hematopoietic Stem Cell Therapy, (E. D. Ball, J. Lister & P. Law, Churchill Livingstone, 2000).

Equivalents

While specific embodiments of the subject invention have been discussed, the above specification is illustrative and not restrictive. Many variations of the invention will become apparent to those skilled in the art upon review of this specification. The appendant claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entireties as if each individual publication or patent was specifically and individually indicated to be incorporated by reference. In case of conflict, the present application, including any definitions herein, will control.

1. A composition comprising a placental stem cell which expresses at least one marker selected from the group consisting of: Thy-1, OCT-4, SOX2, SSEA3, SSEA4, TRA1-60, TRA1-81, Lefty A, FGF-4, Rex-1 and TDGF-1.

2. A composition comprising a placental stem cell which expresses at least two markers selected from the group consisting of: Thy-1, OCT-4, SOX2, SSEA3, SSEA4, TRA1-60, TRA1-81, Lefty A, FGF-4, Rex-1 and TDGF-1.

3. A composition of claim 1 that is ATCC deposit No. ______.

4. A pharmaceutical composition comprising an effective amount of the composition of claim 1 and a pharmaceutically acceptable carrier.

5. A method of making a cardiomyocyte comprising culturing a stem cell of claim 1 in a media that contains an appropriate amount of ascorbic acid 2-phosphate under appropriate conditions and for a sufficient period of time for the stem cell to differentiate into a cardiomyocyte.

6. A cardiomyocyte obtained from the process of claim 5, which expresses at least one marker selected from the group consisting of: MLC-2A, MLC-2V, hANP, cTnT, alpha-actinin, GATA-4 and Nkx 2.5.

7. A cardiomyocyte obtained from the process of claim 5, which expresses at least two markers selected from the group consisting of: MLC-2A, MLC-2V, hANP, cTnT, alpha-actinin, GATA-4 and Nkx 2.5.

8. A cardiomyocyte that is ATCC deposit No. ______.

9. A pharmaceutical composition comprising an effective amount of a cardiomyocyte of claim 6 and a pharmaceutically acceptable carrier.

10. A method of determining whether a test agent is toxic to a cardiomyocyte, comprising contacting the cardiomyocyte of claim 6 with an appropriate amount of the test agent for a time sufficient for a toxic effect on the cardiomyocyte to be detected, and determining whether the test agent has a toxic effect on the cardiomyocyte.

11. A method of determining a metabolic product of a test agent comprising contacting the cardiomyocyte of claim 6 with an appropriate amount of the test agent for a time sufficient for the test agent to be metabolized, and detecting the presence of the metabolized product.

12. A method of making a hepatocyte comprising culturing a stem cell of claim 1 in a media that contains an appropriate amount of dexamethasone, ITS, EGF, FGF-2, FGF-4, FGF-7, HGF, phenobarbital, Type-I collagen or a combination thereof under appropriate conditions and for a sufficient period of time for the stem cell to differentiate into a hepatocyte.

13. A hepatocyte obtained from the process of claim 12, which expresses at least one marker selected from the group consisting of: albumin, CYP1A1, CYP1A2, CYP2B6, CYP2C8, CYP2C9, CYP2D6, CYP3A4, AFP, A1AT, HNF1, HNF4 and C/EBP alpha.

14. A hepatocyte obtained from the process of claim 12, which expresses at least two markers selected from the group consisting of: albumin, CYP1A1, CYP1A2, CYP2B6, CYP2C8, CYP2C9, CYP2D6, CYP3A4, AFP, A1AT, HNF1, HNF4 and C/EBP alpha.

15. A hepatocyte that is ATCC deposit No. ______.

16. A pharmaceutical composition comprising an effective amount of a hepatocyte of claim 13 and a pharmaceutically acceptable carrier.

17. A method of determining whether a test agent is toxic to a hepatocyte comprising contacting the hepatocyte of claim 13 with an appropriate amount of the test agent for a time sufficient for a toxic effect on the hepatocyte to be detected, and determining whether the test agent has a toxic effect on the hepatocyte.

18. A method of determining a metabolic product of a test agent comprising contacting the hepatocyte of claim 13 with an appropriate amount of the test agent for a time sufficient for the test agent to be metabolized, and detecting the presence of the metabolized product.

19. A method of making a pancreatic cell comprising culturing a stem cell of claim 1 in a media that contains an appropriate amount of nicotinamide, dexamethasone, ITS, matrigel or a combination thereof under appropriate conditions and for a sufficient period of time for the stem cell to differentiate into a pancreatic cell.

20. A pancreatic cell obtained from the process of claim 19, which expresses at least one marker selected from the group consisting of: Pax6, Pdx1, insulin, glucagon, and Nkx2.2.

21. A pancreatic cell obtained from the process of claim 19, which expresses at least two markers selected from the group consisting of: Pax6, Pdx1, insulin, glucagon, and Nkx2.2.

22. A pancreatic cell that is ATCC deposit No. ______.

23. A pharmaceutical composition comprising an effective amount of a pancreatic cell of claim 20 and a pharmaceutically acceptable carrier.

24. A method of determining whether a test agent is toxic to a pancreatic cell comprising contacting the pancreatic cell of claim 20 with an appropriate amount of the test agent for a time sufficient for a toxic effect on the pancreatic cell to be detected, and determining whether the test agent has a toxic effect on the pancreatic cell.

25. A method of determining a metabolic product of a test agent comprising contacting the pancreatic cell of claim 20 with an appropriate amount of the test agent for a time sufficient for the test agent to be metabolized, and detecting the presence of the metabolized product.

26. A method of making a neural cell comprising culturing a stem cell of claim 1 in a media that contains an appropriate amount of trans-retinoic acid or FGF-4 under appropriate conditions and for a sufficient period of time for the stem cell to differentiate into a neural cell.

27. A neural cell obtained from the process of claim 26, which expresses at least one marker selected from the group consisting of: GFAP, CNP, beta-tubulin III, Nestin, GAD, NSE, NF-M and MBP.

28. A neural cell obtained from the process of claim 26, which expresses at least two markers selected from the group consisting of: GFAP, CNP, beta-tubulin III, Nestin, GAD, NSE, NF-M and MBP.

29. A neural cell that is ATCC deposit No. ______.

30. A pharmaceutical composition comprising an effective amount of a neural cell of claim 27 and a pharmaceutically acceptable carrier.

31. A method of determining whether a test agent is toxic to a neural cell comprising contacting the neural cell of claim 27 with an appropriate amount of the test agent for a time sufficient for a toxic effect on the neural cell to be detected, and determining whether the test agent has a toxic effect on the neural cell.

32. A method of determining a metabolic product of a test agent comprising contacting the neural cell of claim 27 with an appropriate amount of the test agent for a time sufficient for the test agent to be metabolized, and detecting the presence of the metabolized product.

33. A method of making a vascular endothelial cell comprising culturing a stem cell of claim 1 in a media that contains matrigel under appropriate conditions and for a sufficient period of time for the stem cell to differentiate into a vascular endothelial cell.

34. A vascular endothelial cell obtained from the process of claim 33, which expresses the FLT-1 marker.

35. A vascular endothelial cell obtained from the process of claim 33, which has physical characteristics of cells shown in FIG. 10.

36. A vascular endothelial cell that is ATCC deposit No. ______.

37. A pharmaceutical composition comprising an effective amount of a vascular endothelial cell of claim 34 and a pharmaceutically acceptable carrier.

38. A method of determining whether a test agent is toxic to a vascular endothelial cell comprising contacting the vascular endothelial cell of claim 34 with an appropriate amount of the test agent for a time sufficient for a toxic effect on the vascular endothelial cell to be detected, and determining whether the test agent has a toxic effect on the vascular endothelial cell.

39. A method of determining a metabolic product of a test agent comprising contacting the vascular endothelial cell of claim 34 with an appropriate amount of the test agent for a time sufficient for the test agent to be metabolized, and detecting the presence of the metabolized product.
